(19) United States
(12) Patent Application Publication
(10) Pub. No.: US 2025/0292873 A1
(43) Pub. Date: Sep. 18, 2025

(54) DEVELOPMENT SUPPORT SYSTEM AND DEVELOPMENT SUPPORT DEVICE OF MEMBRANE REACTOR

(71) Applicant: eSep Inc., Souraku-gun, Kyoto (JP)

(72) Inventor: Kenichi SAWAMURA, Souraku-gun, Kyoto (JP)

(21) Appl. No.: 18/575,821

(22) PCT Filed: Jan. 16, 2023
(86) PCT No.: PCT/JP2023/001062

§ 371 Date: Dec. 30, 2023

(30) Foreign Application Priority Data
Feb. 9, 2022 (JP) 2022-018981

(51) Int. Cl. G16C 20/10; G16C 20/80; G16C 20/90

(52) U.S. Cl. CPC G16C 20/10; G16C 20/80; G16C 20/90

(57) ABSTRACT

Provided are a development support system and a development support device that allow many users to easily perform a simulation related to a membrane reactor. A client device performs a step of once equally dividing a series of reaction separation process steps P into ten billion unit cells 2, a step of dividing a section from an inlet 4 to an outlet 6 of the series of reaction separation process steps P into a plurality of section divisions 1 to 6 that can be represented by the number of unit cells 2, and bundling the unit cells 2 included in the divided sections into 1,000, 90, 90, 90, 90, and 9,990, and a step of calculating a mass balance of the unit cells 2 by sequential calculation from the inlet 4 to the outlet 6 of the series of reaction separation process steps.

### TECHNICAL FIELD

The present invention relates to, for example, a development support (assistance) system and a development support (assistance) device of a membrane reactor with a membrane reaction.

### BACKGROUND ART

Patent Literature 1 (paragraph 0074 and the like) described below discloses performing chemical engineering simulation on a membrane reactor that realizes membrane permeation and reaction. In addition, Patent Literature 1 discloses that the inventors have independently created a routine using a VBA code.

### CITATION LIST

### Patent Literature

Patent Literature 1: JP 2018-008940 A

### SUMMARY OF INVENTION

### Technical Problem

Meanwhile, Patent Literature 1 discloses that a commercially available process simulator is not packaged as a device for a separation membrane reactor. Such circumstances have not largely changed even in recent years. Still, a user of the separation membrane reactor often performs simulation while devising software (application program) for fluid numerical analysis.

Patent Literature 1 also discloses that a user of the separation membrane reactor creates a routine for chemical engineering simulation by himself/herself. However, the user of the separation membrane reactor does not necessarily have sufficient skill (skill) in programming. Therefore, design and selection of the separation membrane reactor are not usually easy.

An object of the present invention is to provide a development support system and a development support device that allow many users to easily perform a simulation related to a membrane reactor.

### Solution to Problem

(1) According to an aspect of the present invention, there is provided a development support system including a service-provider-side terminal device and a user-side terminal device that are communicably connected, in which the service-provider-side terminal device determines whether or not a user of the user-side terminal device is a user satisfying a predetermined permission condition in a case where there is a request for use of a predetermined development support program from the user-side terminal device, and makes the development support program available for use to the user when it is determined that the user is the user satisfying the permission condition, and at least a part of the development support program is described using an extended function language for giving an extended function to an application program with a calculation function installed in the user-side terminal device to cause the user-side terminal device to analyze a flow step of a membrane reactor that simultaneously performs a chemical reaction and membrane separation.

(2) According to another aspect of the present invention, there is provided a development support device including a service-provider-side terminal device and a user-side terminal device that are communicably connected, in which the service-provider-side terminal device determines whether or not a user of the user-side terminal device is a user satisfying a predetermined permission condition in a case where there is a request for use of a predetermined development support program from the user-side terminal device, and makes the development support program available for use to the user when it is determined that the user is the user satisfying the permission condition, and at least a part of the development support program is described using an extended function language for giving an extended function to an application program with a calculation function installed in the user-side terminal device to cause the user-side terminal device to analyze a flow step of a membrane reactor that simultaneously performs a chemical reaction and membrane separation.

### Advantageous Effects of Invention

According to the present invention, it is possible to provide a development support system and a development support device of a membrane reactor that allow many users to easily perform a simulation related to a membrane reactor.

### BRIEF DESCRIPTION OF DRAWINGS

FIG. 1 is an explanatory diagram illustrating a relationship between a membrane reactor and a unit cell.

FIG. 2 is a schematic configuration diagram illustrating one embodiment of the invention according to a development support system.

FIG. 3(a) is a block diagram schematically illustrating a configuration of a server device, and FIG. 3(b) is a block diagram schematically illustrating a configuration of a client device.

FIG. 4 is a flowchart illustrating a basic configuration of the invention according to a development support method.

FIG. 5 is a flowchart illustrating a basic configuration of an authentication step.

FIG. 6 is an explanatory diagram schematically illustrating a data configuration of user management information.

FIG. 7 is an explanatory diagram schematically illustrating a data configuration of analysis history information.

FIG. 8 is an explanatory diagram illustrating an example of a parameter input screen.

FIG. 9 is an explanatory diagram illustrating an example of an input cell 1 column.

FIG. 10 is an explanatory diagram illustrating an example of an input cell 2 column.

FIG. 11(a) is an explanatory diagram illustrating a part of an example of an input cell 3 column, and FIG. 11(b) is an explanatory diagram illustrating another part of the example of the input cell 3 column.

FIG. 12 is an explanatory diagram illustrating an example of a result display cell 1 column.

FIG. 13(a) is an explanatory diagram illustrating a part of an example of a result display cell 2 column, and FIG. 13(b) is an explanatory diagram illustrating another part of the example of the result display cell 1 column.

FIG. 14 is an explanatory diagram schematically illustrating a membrane reaction module.

FIG. 15 is an explanatory diagram illustrating an example of the membrane reactor.

FIG. 16 is an explanatory diagram illustrating another example of the membrane reactor.

FIG. 17 is an explanatory diagram illustrating a relationship among a series of reaction separation process steps, unit cells, and section divisions.

FIG. 18 is a flowchart schematically illustrating the contents of calculation display processing.

FIG. 19 is an explanatory diagram illustrating an example of an analysis result display screen.

FIG. 20 is an explanatory diagram illustrating an example of a calculation result in a case where a parameter value is changed.

FIG. 21 is an explanatory diagram illustrating an example of input related to a parameter value.

FIGS. 22(a) and 22(b) are explanatory diagrams illustrating calculation results when calculation is performed using divergence suppression processing for parameter input of FIG. 21, and FIG. 22(c) is an explanatory diagram illustrating an analysis result based on the calculation results.

FIGS. 23(a) and 23(b) are explanatory diagrams illustrating calculation results in a case where calculation is performed on the parameter input of FIG. 21 without using the divergence suppression processing, and FIG. 23(c) is an explanatory diagram illustrating an analysis result based on the calculation results.

FIG. 24 is an explanatory diagram illustrating a parameter input screen before executing a back calculation step.

FIG. 25 is an explanatory diagram illustrating the parameter input screen after execution of the back calculation step.

FIG. 26 is a flowchart schematically illustrating another embodiment 1 related to the divergence suppression processing.

FIG. 27 is a flowchart schematically illustrating another embodiment 2 related to the divergence suppression processing.

### DESCRIPTION OF EMBODIMENTS

Hereinafter, preferred embodiments of the present invention will be described in detail with reference to the drawings. Note that, in the present embodiment and the drawings according to the present embodiment, components denoted by the same reference numerals have similar structures or functions.

### <Example of Membrane Reactor to Be Simulated>

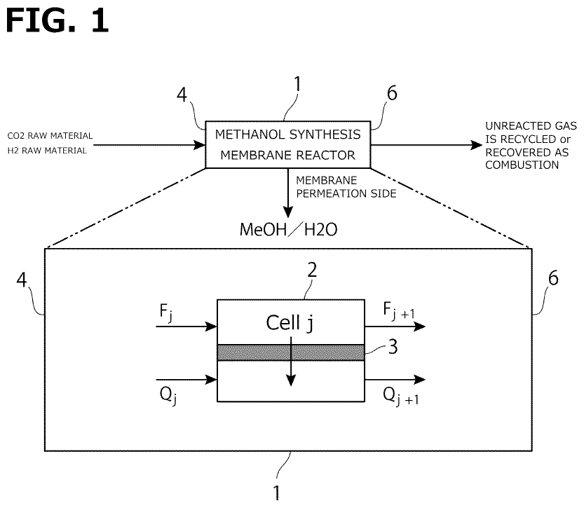

FIG. 1 schematically illustrates a flow step of a membrane reactor (methanol synthesis membrane reactor) 1 to be simulated. In the example of FIG. 1, the membrane reactor 1 is for synthesizing methanol (MeOH) from carbon dioxide (CO₂) and hydrogen (H₂) using a separation membrane 3. A CO₂ (carbon dioxide) raw material and a H₂ (hydrogen) raw material are supplied to the membrane reactor 1, and MeOH (methanol) and H₂O (water) are separated on the membrane permeation side (permeation side of the separation membrane 3) of the membrane reactor 1. The unreacted gas is recycled or recovered as combustion heat.

As will be described in detail later, a flow step (a series of reaction separation process steps) in the membrane reactor 1 is equally divided (equal division) into a large number (For example, 10,000 to ten billion or the like) of unit cells 2. The unit cell 2 is used to derive a theoretical formula for simulation. The notation of “cell j” in FIG. 1 means an arbitrary unit cell 2 among a large number of equally divided unit cells 2. In the simulation, calculation processing (sequential calculation) and sequential analysis are executed for each of the unit cells 2, and a phenomenon in a series of reaction separation process steps (steps related to the process of chemical reaction and membrane separation) is clarified.

Here, the “sequential calculation” can be described as performing the calculation of Fⱼ₊₁ and Qⱼ₊₁ related to the next (subsequent) unit cellⱼ₊₁ one after another using Fj and Qj obtained in the unit cell j−1 immediately before (preceding) the unit cell j in FIG. 1. Further, “sequential analysis” can be described as obtaining the concentration and partial pressure of a fluid (gas in this case) in the unit cell 2.

The division of the unit cell 2 may be division along a spatial axis or division along a time axis. When the series of reaction separation process steps is divided along the spatial axis, the unit of the division axis of the unit cell 2 is length. When the division is performed along the time axis, the unit of the division axis of the unit cell 2 is time.

### <Basic Configuration of Development Support System 10>

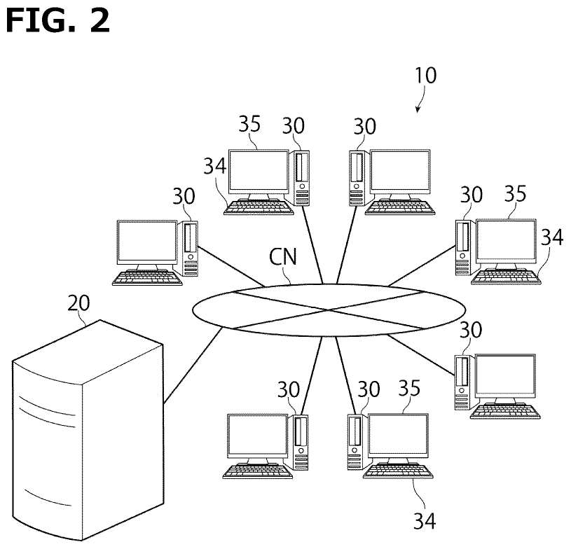

FIG. 2 schematically illustrates a development support system 10 according to one embodiment of the invention. A development support system 10 includes server device 20 and a plurality of client devices 30, . . . . The server device 20 and the client device 30 are communicably connected to each other via a communication network CN. Examples of the communication network CN include a network (including a so-called cloud) connected to each other via the Internet, a LAN, a WAN, a public telephone line, a base station, a mobile communication network, a gateway, or the like.

In the development support system 10, a supplier of a product of the membrane reactor 1 provides a consumer (hereinafter, referred to as a “user”) who examines specifications of the membrane reactor 1 with a service for supporting development of the membrane reactor 1. Examples of the user include a business operator, a research institution, or the like which is going to purchase the product of the membrane reactor 1.

The main content of the service is provision of an application program (also referred to as a “simulation program”, a “simulator”, a “development support program”, and the like.) for simulation related to the membrane reactor 1. Details of the simulation related to the membrane reactor 1 will be described later.

The server device 20 is a computer device operated by the supplier, and the client device 30 is a computer device used by a user. As the server device 20 and the client device 30, general computer devices can be used.

For example, as the server device 20, it is possible to use a terminal device such as a personal computer (hereinafter referred to as “PC”) in addition to a so-called server-dedicated device. As the client device 30, a PC used by the user on a daily basis can be used.

As the PC used as the client device 30, in addition to a desktop type, a notebook type or a tablet type can also be adopted. The client device 30 may be a smartphone or the like as long as it can execute a simulation program to be described later.

### <<Configuration of Server Device 20>>

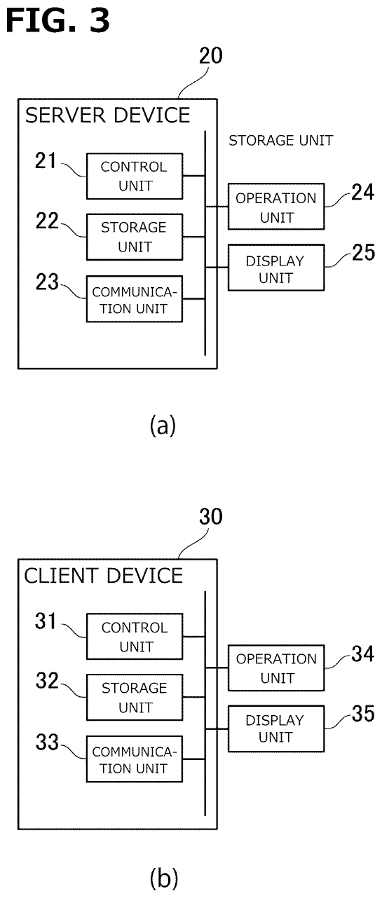

FIG. 3(a) schematically illustrates an internal configuration of the server device 20 and peripheral devices. The server device 20 internally includes a control unit 21, a storage unit 22, a communication unit 23, and the like, and includes an operation unit 24, a display unit 25, and the like as peripheral devices.

Although not illustrated, the control unit 21 includes a central processing unit (CPU), a read only memory (ROM), a random access memory (RAM), and the like. The CPU of the control unit 21 develops and executes various computer programs stored in the ROM and the storage unit 22 on the RAM. The control unit 21 may be any processing circuit or arithmetic circuit including a plurality of CPUs, multi-core CPUs, graphics processing units (GPUs), microcomputers, volatile or nonvolatile memories, and the like.

The storage unit 22 is a nonvolatile storage unit including a semiconductor memory such as a read only memory (ROM) or a random access memory (RAM) that stores various types of information, a hard disk drive (HDD), a solid state drive (SSD), or the like. The storage unit 22 stores an operating system program, a driver program, an application program, data, and the like used for processing in the processor (here, the control unit 21).

The program stored in the storage unit 22 may be provided by a non-transitory recording medium (not illustrated) in which the program is readably recorded. Examples of the recording medium include portable memories such as a CD-ROM, a universal serial bus (USB) memory, a secure digital (SD) card, a micro SD card, and a compact flash (registered trademark). In this case, the control unit 21 reads the program from the recording medium using a reading device (not illustrated), and installs the read program in the storage unit 22.

The program stored in the storage unit 22 may be provided by communication via the communication unit 23. In this case, the control unit 21 acquires the program through the communication unit 23 and installs the acquired program in the storage unit 22.

The communication unit 23 includes an interface for communicating with the client device 30 through the communication network CN. The communication unit 23 executes data communication according to a predetermined communication protocol. In a case where information to be transmitted to the client device 30 is input from the control unit 21, the communication unit 23 transmits the input information to the client device 30. The communication unit 23 outputs information from the client device 30 received through the communication network CN to the control unit 21.

The operation unit 24 and the display unit 25 are connected to the server device 20. The operation unit 24 is, for example, an operation input unit such as a keyboard, a mouse, or a touch panel. Although only one operation unit 24 is illustrated in FIG. 3(a), the operation unit 24 comprehensively illustrates these operation input unit. The display unit 25 is a display unit such as a general display device. Note that either the operation unit 24 or the display unit 25 may not be connected to the server device 20, and both the operation unit 24 and the display unit 25 may not be connected.

The server device 20 has a function (analysis management function) of transmitting a simulation program, permitting use, and the like to the client device 30. Furthermore, the server device 20 also has a function (billing management function) of confirming whether or not a usage fee related to the simulation program has been paid.

When receiving access from the client device 30, the server device 20 performs user authentication based on, for example, a user ID and a password. When the authentication of the user who has paid a usage fee succeeds, the server device 20 permits the use of the simulation program and starts providing the service to the client device 30. Note that a server device for billing management may be separately provided, and the server device for analysis management and the server device for billing management may be connected via the communication network CN.

### <<Configuration of Client Device 30>>

FIG. 3(b) schematically illustrates an internal configuration of the client device 30 and peripheral devices. The client device 30 internally includes a control unit 31, a storage unit 32, a communication unit 33, and the like, and includes an operation unit 34, a display unit 35, and the like as peripheral devices. Among these units, as the control unit 31, the storage unit 32, the operation unit 34, and the display unit 35, the same units as the control unit 21, the storage unit 22, the operation unit 24, and the display unit 25 of the server device 20 illustrated in FIG. 3(a) can be adopted, and thus the descriptions thereof will be omitted here.

The communication unit 33 of the client device 30 includes an interface for communicating with the server device 20 through the communication network CN. The communication unit 33 executes data communication according to a predetermined communication protocol. In a case where information to be transmitted to the server device 20 is input from the control unit 31, the communication unit 33 transmits the input information to the server device 20. The communication unit 33 outputs information from the server device 20 received through the communication network CN to the control unit 31.

In the client device 30, an application program (a web browser or the like) for accessing the server device 20 is installed. The user accesses the server device 20 via the client device 30. When the authentication by the server device 20 is successful, the user can receive a predetermined service from a service provider via the server device 20.

Here, the development support system 10 according to the present embodiment can also be regarded as a development support device including the server device 20 and the client device 30. In addition, each of the server device 20 and the client device 30 can be regarded as a development support device.

### <Basic Configuration of Development Support Method>

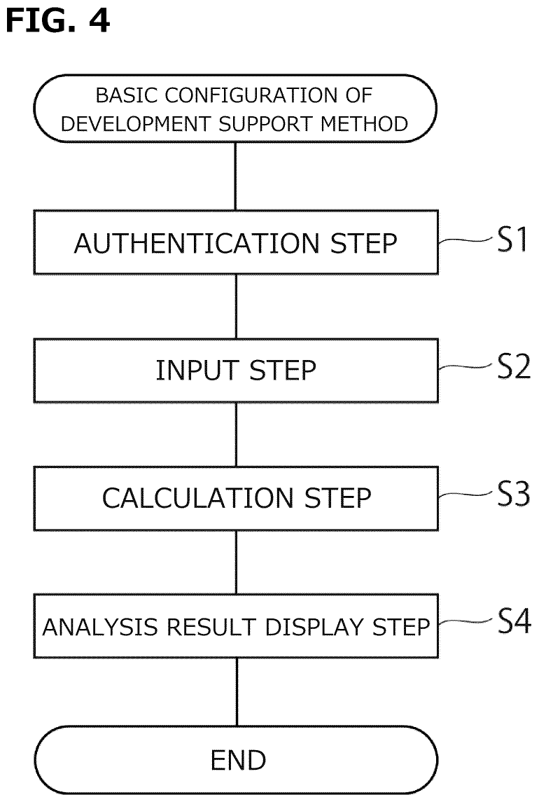

FIG. 4 illustrates a basic configuration of a development support method executed using the development support system 10 illustrated in FIG. 2. The development support method of the present embodiment includes an authentication step S1, an input step S2, a calculation step S3, and an analysis result display step S4.

In the authentication step S1, the user information from the client device 30 is authenticated, and the use of the simulation program by the user is permitted. In the input step S2, conditions (parameter values) necessary for simulation of the membrane reactor 1 are input. In the calculation step S3, a chemical reaction calculation and a membrane permeation separation calculation are executed based on the input conditions. In the analysis result display step S4, various graphs are created and displayed on the basis of the calculation in the calculation step S3.

In the present embodiment, the simulation program is performed using an application program installed in the client device 30. As the application program, one for spreadsheet calculation (hereinafter referred to as “spreadsheet software”) is used.

In general, in a PC used for business use or personal use, spreadsheet software is installed in addition to an application program for text creation and the like. Further, some spreadsheet software enables a user to extend functions using a specific programming language (extended function language). In the present embodiment, the chemical reaction and the membrane separation are calculated using a general and versatile application program such as spreadsheet software instead of a dedicated application program specialized for numerical analysis of fluids and the like.

As the spreadsheet software, various kinds of software can be used as long as it can perform calculation necessary for analysis of a series of reaction separation process steps in the membrane reactor 1. In the present embodiment, Excel (trade name) of Microsoft Corporation is used as spreadsheet software. In order to simulate the membrane reactor 1 by function expansion, Visual Basic for Applications (VBA) is used as an extended function language. Hereinafter, each of steps S1 to S4 will be described.

### <Authentication Step S1>

### <<Flow of Authentication Work>>

In the authentication step S1, when a service-provider-side terminal device (here, the server device 20 (FIG. 2)) receives a request for use of a predetermined simulation program from a user-side terminal device (here, the client device 30), whether or not the user of the user-side terminal device is a user satisfying a predetermined permission condition (that a fee has been paid, or the like) is determined.

In a case where the user satisfies the permission condition, the simulation program becomes available.

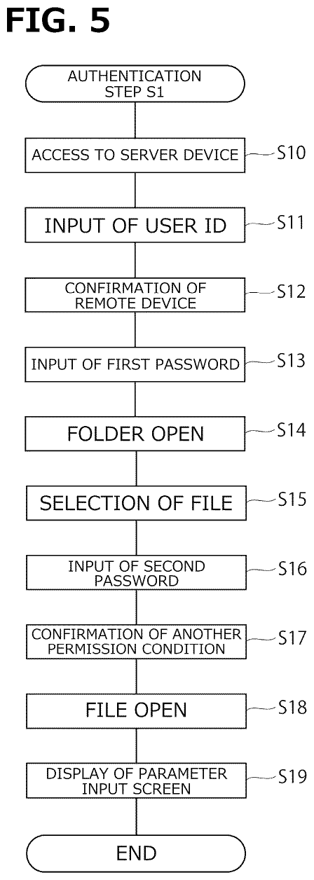

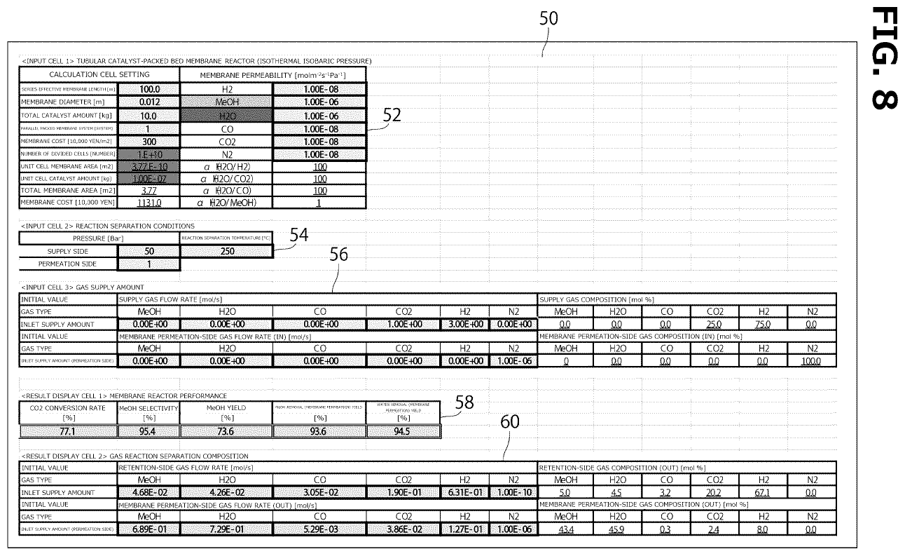

FIG. 5 sequentially illustrates main work contents in the authentication step S1. In the authentication step S1, the following steps are performed: access to the server device 20 (S10), input of a user ID (identification number) (S11), confirmation of a remote device (S12), input of a first password (S13), folder open (S14), selection of a file (S15), input of a second password (S16), confirmation of another permission condition (S17), file open (S18), and display of a parameter input screen (interface screen, FIG. 8) (S19).

In the access to the server device (S10), the user accesses the server device 20 using a web browser in the client device 30 (FIGS. 2 and 3(a)). In the client device 30, the user performs an operation for requesting use of the simulation program.

Although the screen display in the authentication step S1 is not illustrated, in the step of inputting the user ID (S11), an ID input screen is popped up on the display unit 35 of the client device 30 (FIGS. 2 and 3(b)). When the user inputs an ID (user ID) assigned to the user in advance on the ID input screen via the operation unit 34 and operates a button (also referred to as a “next button” or a “forward button”) for instructing transition to the next step, confirmation of the remote device (S12) is performed.

In the step of remote device confirmation (S12), an ID display screen (client ID display screen) is popped up on the display unit 35 of the client device 30. When the user selects the ID of the client device 30, the processing proceeds to the next step, and the first password is input (S13).

In the input of the first password (S13), a pop-up display of a first password input screen is performed on the display unit 35 of the client device 30. When the user inputs an appropriate first password assigned to the user in advance on the first password input screen via the operation unit 34 and operates the “next button”, the processing proceeds to the step of folder opening (S14).

In the folder open (S14), a folder of the simulation program is displayed. At this time, a folder of the simulation program is automatically opened and displayed so that the user can immediately visually recognize an icon of the simulation program. Note that the folder may be displayed in a closed state, and the user may perform an operation (for example, a double click) for opening the folder to open the folder.

When the user selects a file displayed in the opened folder (S15) and performs an operation to open the file (for example, double click), the processing proceeds to the input of the second password (S16). In the input of the second password (S16), a second password input screen is popped up on the display unit 35 of the client device 30. When the user inputs an appropriate second password assigned to the user in advance on the second password input screen via the operation unit 34 and operates the “next button”, the processing proceeds to confirmation of another permission condition (S17).

Here, the second password may be a part of continuous characters or symbols of the first password so that the user can easily manage the second password.

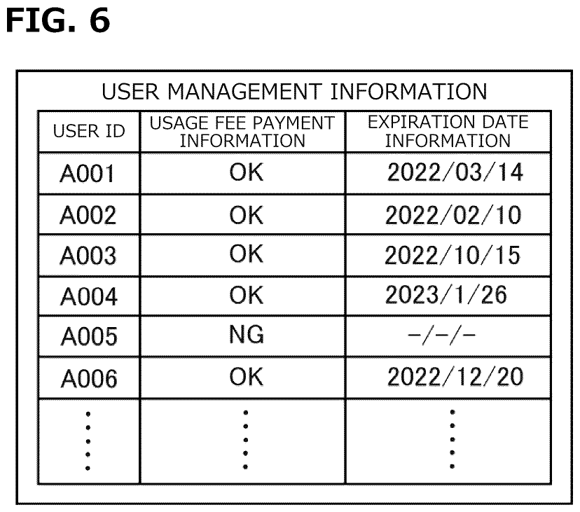

In the confirmation of the other permission conditions (S17), it is confirmed whether or not the usage fee of the simulation program has been properly paid for the user. With respect to the information of the user ID transmitted from the client device 30, the server device 20 confirms whether or not the fee for service provision is properly paid with reference to user management information (FIG. 6) to be described later.

When the server device 20 determines that the fee imposed on the use of the simulation program has been properly paid, the server device 20 transmits information (use permission information) indicating that the use of the simulation program is permitted to the client device 30. When the use permission information is received, the login of the user is completed in the client device 30, and the processing proceeds to the step of file opening (S17).

As the processing proceeds to the step of file opening (S17), the user can use the simulation program. In the present embodiment, the simulation program is the spreadsheet software installed in the client device 30 and to which an extended function is added. In the step of file opening (S17), execution of the spreadsheet software is started.

When the execution of the simulation program is started in this manner, the processing proceeds to the step of displaying the input screen (S18). In the display of the input screen (S18), a parameter input screen 50 as illustrated in an example in FIG. 8 is displayed, and the user can perform simulation. The parameter input screen 50 is provided in a work sheet of the spreadsheet software, and the contents of the parameter input screen 50 will be described later.

The information of the user ID, the ID of the client device 30, the first password, and the second password described above is referred to as information registered in the server device 20. When these pieces of information match the registered information, the simulation is permitted. However, in a case where even one of these pieces of information cannot be confirmed in the server device 20, simulation by the client device 30 is not permitted, and error display is performed. In this case, the user cannot log in and cannot proceed to the subsequent processing.

In a case where the login is performed, the user ID, the first password, and/or the second password at that time may be saved in the client device 30, and thereafter, input of these pieces of information may be omitted.

The modes of the ID input screen, the display screen of the ID of the client device 30, the first password input screen, and the second password input screen can be different depending on the type and version of the operating system (OS) program installed in the client device 30.

In addition, in a case where the second password input screen is displayed, when the user operates a so-called “read-only button” without inputting the second password, the simulation may be permitted. The “read-only button” mentioned herein is a button image that can be operated by the user to indicate his/her intention not to write (modify) in the simulation software to be used.

Furthermore, the authentication mode between the server device 20 and the client device 30 can be variously set and changed as long as the use of the simulation program can be permitted only to an appropriate user.

For example, each time the user inputs or operates the information of the user ID, the ID of the client device 30, the first password, and the second password, these pieces of information may be transmitted from the client device 30 to the server device 20. In the server device 20, every time these pieces of information are received, it is possible to refer to the registered information one by one, and in a case where a plurality of (some or all) pieces of information are prepared, it is possible to collectively refer to the plurality of pieces of information as the registered information.

Furthermore, for example, when the user ID is input (S11), the information of the user ID is transmitted from the client device 30 to the server device 20, and the server device 20 can transmit the information of the registered first password and second password corresponding to the user ID to the client device 30. In this case, authentication can be performed in the client device 30.

The result of the authentication performed by the client device 30 can be transmitted to the server device 20. The server device 20 can store the information related to the authentication result received from the client device 30, and can perform determination of the payment status of the fee (determination related to confirmation of other permission conditions (S17)).

### <<Association of Information>>

The authentication of the user in the authentication step S1 can be performed on the basis of the user ID and various types of information (user management information) associated with the user ID. FIG. 6 illustrates an example of a combination of the user management information. FIG. 6 illustrates “usage fee payment information” and “expiration date information” as information associated with the user ID.

In the example of FIG. 6, six user IDs “A001” to “A006” are illustrated, and the other user IDs are not illustrated. The “usage fee payment information” in FIG. 6 is information indicating whether or not the usage fee has been paid. In the example of FIG. 6, “OK” indicates that the payment has been made, and “NG” indicates that the payment has not been made. For example, the users with the user IDs “A001” to “A004” and “A006” have already paid the usage fee, and the user with the user ID “A005” has not paid the usage fee.

The “expiration date information” is information indicating a last date of a period in which the user who has paid the usage fee can use the simulation program. In the example of FIG. 6, the user with the user ID “A001” can use the simulation program until Mar. 14, 2022, and the user with the user ID “A002” can use the simulation program until Feb. 10, 2022.

In addition, for a user who cannot use the simulation program such as “A005”, the user cannot log in to the site for providing the simulation program and cannot use the simulation program. In a case where the login is not possible as described above, a display indicating that the login is not possible (error display) is performed, and the display timing at this time can be a timing at which, in the input of the first password (S13) or the input of the second password (S16), it cannot be confirmed that the usage fee has been paid, and the authentication fails.

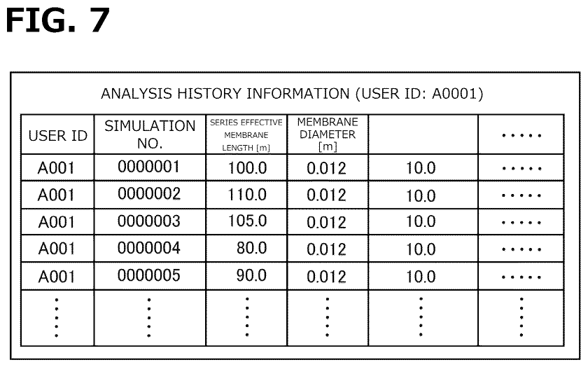

Furthermore, FIG. 7 illustrates analysis history information as an example of information associated with the user ID. In the example of FIG. 7, “user ID”, “simulation No.”, “series effective membrane length”, “membrane diameter”, and “total catalyst amount” are exemplified as the information included in the analysis history information. Among these pieces of information, “simulation No.” is a number assigned to each simulation performed using a specific user ID (here, “A001”).

For example, a simulation with “simulation No.” of “0000001” indicates the first simulation by the user of “A001”. In addition, the simulation with “simulation No.” of “0000005” indicates the fifth simulation by the user of “A001”.

In addition, the “series effective membrane length”, the “membrane diameter”, and the “total catalyst amount” are values input by the user in the simulation. In the example of FIG. 7, each input value in a plurality of simulations corresponding to one user ID (A001) is illustrated.

For example, the user of “A001” inputs values of “100.0”, “0.012”, and “10.0” to the “series effective membrane length”, the “membrane diameter”, and the “total catalyst amount”, respectively, in the first simulation. Further, this user sets the “series effective membrane length”, the “membrane diameter”, and the “total catalyst amount” to “90.0”, “0.012”, and “10.0”, respectively, in the fifth simulation.

Here, the information such as the “series effective membrane length”, the “membrane diameter”, and the “total catalyst amount” is information input by the user. There are other types of information input by the user, but the type and content of the input information will be described later. In each simulation, a predetermined calculation is performed based on these input values, and a calculation result is displayed. The type and content of the calculation result will also be described later. The calculation result can also be stored in association with the information of the “user ID” or the “simulation No.”.

Various pieces of information illustrated in FIGS. 6 and 7 are stored in a database (not illustrated) as table data. In the database, various types of information are stored in association with each “user ID” or “simulation No.”.

The database may be constructed in the storage unit 22 of the server device 20, or may be constructed in a storage unit (such as a storage unit of the database server) provided separately from the server device 20 and connected to the communication network CN. The database and the server device 20 (management server) may be disposed in the same premises, or may be disposed in a building or a region away from each other.

### <Input Step S2>

### <<Overall Configuration of Parameter Input Screen 50>>

In the input step S2, the input of numerical values (parameters) necessary for simulation is received for each item (each variation factor) displayed on the parameter input screen 50 (FIG. 8). As illustrated in FIG. 8, the parameter input screen 50 includes three input columns (here, input cell 1 column 52, input cell 2 column 54, and input cell 3 column 56), two result display columns (here, result display cell 1 column 58 and result display cell 2 column 60), and the like.

Each of the input cell 1 column 52, the input cell 2 column 54, and the input cell 3 column 56 is provided with a plurality of input cells (cell groups) as described later. The user can input a numerical value (parameter value) of a variation factor used for simulation to each of the input cells of the input cell 1 column 52, the input cell 2 column 54, and the input cell 3 column 56.

In the present embodiment, the input cell that can be input by the user is highlighted with a unique color (may be shaded) so that the user can distinguish the input cell from cells other than the input cell. In addition, a “reflection cell” and a “result display cell” to be described later are also displayed with a unique color (may be shaded) so as to be distinguishable from others. Furthermore, in addition to the input cell, the name of the variation factor is indicated on the left side or the upper side of the “reflection cell” or the “result display cell”.

The input cell 1 column 52 is used to input parameters related to membrane reactor characteristics. In the example of FIG. 8, a character string indicating the type of the membrane reactor 1 (here, “tubular catalyst-packed bed membrane reactor (isothermal isobaric pressure)”) is displayed as the title (title) in the input cell 1 column 52. Similarly, the input cell 2 column 54 is used for inputting parameters related to “reaction separation conditions”. The input cell 3 column 56 is used for inputting a parameter related to a “gas supply amount”.

Each of the result display cell 1 column 58 and the result display cell 2 column 60 is provided with a plurality of result display cells (cell groups) as described later. In each result display cell, the result of numerical calculation performed using the numerical values input in the input cell 1 column 52, the input cell 2 column 54, and the input cell 3 column 56 is displayed. The result display cell 1 column 58 is used to display a result related to “membrane reactor performance”. The result display cell 2 column 60 is used for result display related to a “gas reaction separation composition”.

When the result of the numerical calculation is displayed in the result display cell 1 column 58 and the result display cell 2 column 60, the result display may be performed immediately when the input to the input cell 1 column 52, the input cell 2 column 54, and the input cell 3 column 56 is performed. Alternatively, an operation button or a menu such as “execution” may be provided, and the calculation may be started by a user's operation.

Here, in the present embodiment, the term “cell” is used for each display column provided in the work sheet of the spreadsheet software and a physical model (unit cell) obtained by subdividing a series of reaction separation process steps in the membrane reactor 1. In order to distinguish them, the “cell” which is the display column of spreadsheet software is appropriately referred to as an “input cell”, a “reflection cell”, a “result display cell”, and the like, and the “cell” in a series of reaction separation process steps is appropriately referred to as the “unit cell”.

### <<Input Cell 1 Column 52>>

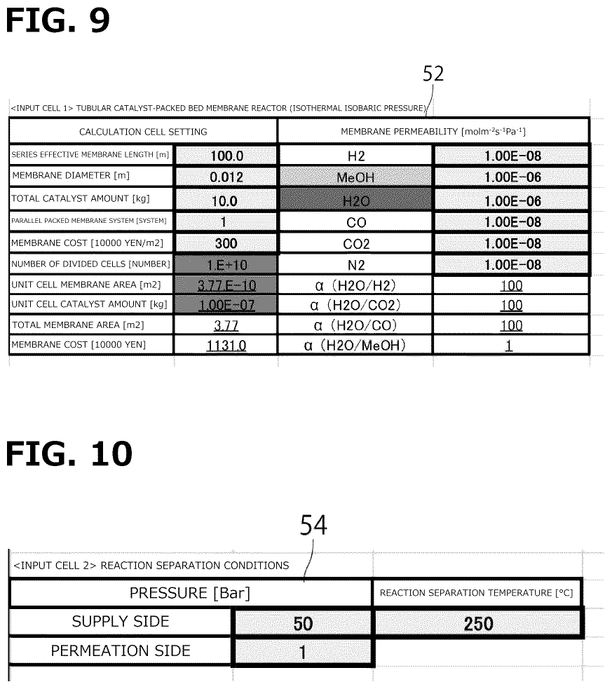

FIG. 9 is an enlarged view of input cell 1 column 52 in FIG. 8.

In the input cell 1 column 52, a column of “calculation cell setting” and a column of “membrane permeability” are provided. Among these, in the column of “calculation cell setting”, columns of a “series effective membrane length”, a “membrane diameter”, a “total catalyst amount”, a “parallel packed membrane system”, a “membrane cost”, the “number of divided cells”, a “unit cell membrane area”, a “unit cell catalyst amount”, a “total membrane area”, and a “membrane cost”, which are variation factors, are provided. Among these, the variation factors that can input the parameters are the “series effective membrane length”, the “membrane diameter”, the “total catalyst amount”, the “parallel packed membrane system”, and the “membrane cost”.

By inputting a parameter to some of the variation factors (for example, “series effective membrane length”, “membrane diameter”, and the like), values of some other variation factors (for example, “total membrane area” and the like) are automatically calculated and displayed in corresponding cells (referred to as “reflection cell”) in the input cell 1 column 52. For example, the “total membrane area” is calculated by inputting parameters (“100.0” and “0.012” in the example of FIG. 9) to the “series effective membrane length” and the “membrane diameter”, and the calculated value (here, “3.77”) is displayed in the corresponding cell (reflection cell).

Here, the “series effective membrane length” and the “total membrane area” are variation factors related to the range of the series of reaction separation steps. The range of the series of reaction separation steps is determined by the form (arrangement and number of membrane reaction modules, and the like) of the membrane reactor 1 and the connection mode (series, parallel, combination of series and parallel, and the like) of the membrane reaction modules in the membrane reactor 1. Therefore, the range of the series of reaction separation steps is determined by the user inputting the “membrane diameter” related to the “series effective membrane length” and the “total membrane area”.

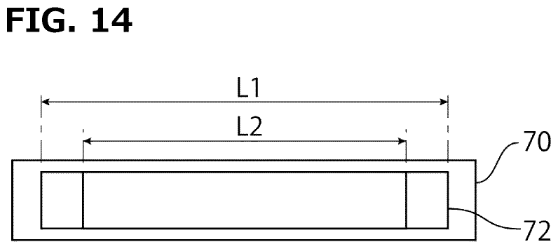

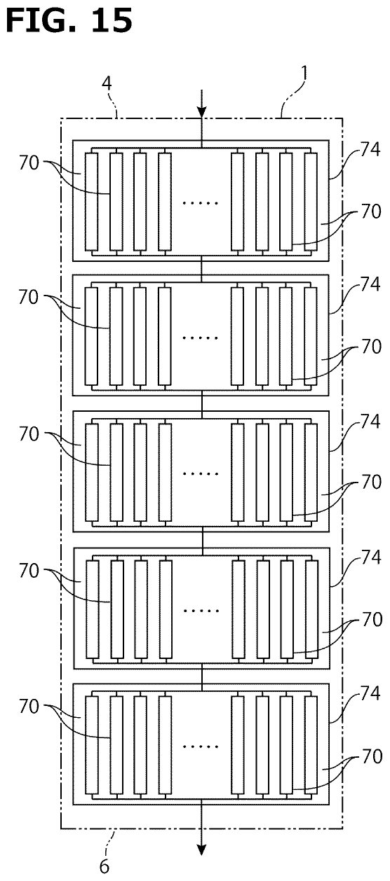

For example, as schematically illustrated in FIG. 14, it is assumed that in one membrane reaction module 70, a length L1 of a separation membrane element 72 is 0.8 m, and a length (effective length) L2 to be effectively used among them is 0.7 m. As illustrated in FIG. 15, it is assumed that a plurality of (31 in this case) membrane reaction modules 70 are used for one container 74. Further, the membrane reaction modules 70 are connected in parallel, and five containers 74 are connected in series to constitute the membrane reactor 1.

In this case, the “series effective membrane length” is 0.7 [m]×5=3.5 [m]. In addition, a width (membrane width) of the membrane can be calculated by using the value input in the “membrane diameter”, and a membrane area can be calculated by using the values of the membrane width and the “series effective membrane length”. Furthermore, an effective membrane area (“total membrane area”) can be calculated by the product of the membrane area of one system in series and the “parallel packed membrane system” using the value of the “parallel packed membrane system” (“31” in this case).

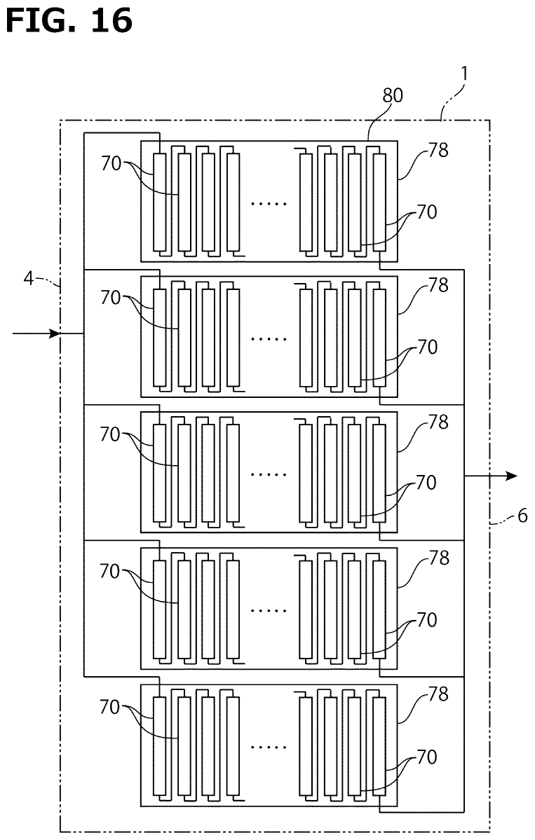

As illustrated in FIG. 16, the plurality of membrane reaction modules 70 may be used in series. In the example illustrated in FIG. 16, 31 membrane reaction modules 70 are connected in series in one container 78. It is assumed that five containers 78 of this type are connected in parallel to constitute the membrane reactor 1. In this case, the “series effective membrane length” is 0.7 [m]×31=21.7 [m]. Furthermore, the value of the “parallel packed membrane system” is “5”, and the effective membrane area (“total membrane area”) can be calculated using these values.

As described above, the “series effective membrane length” and the “membrane diameter” are variation factors for defining the range of a series of reaction separation steps. Further, the “series effective membrane length” is determined by the form of the membrane reactor and the connection mode of the membrane reaction module. The form of the membrane reactor and the connection mode of the membrane reaction module are not limited to those illustrated in FIGS. 15 and 16, and can be variously changed according to the needs of the user.

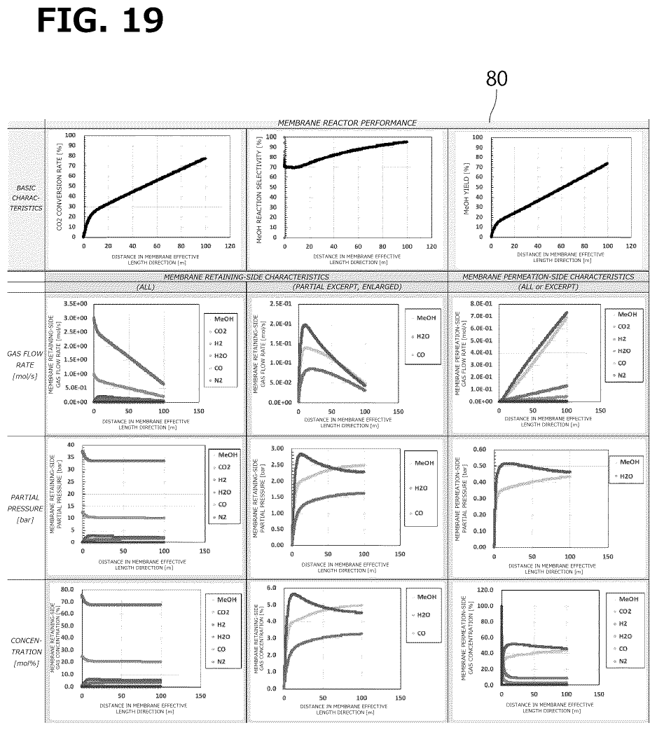

In the input cell 1 column 52 illustrated in FIG. 9, columns of “H₂”, “MeOH”, “H₂O”, “CO”, “CO₂”, “N₂”, “a (H₂O/H₂)”, “a (H₂O/CO₂)”, “a (H₂O/CO)”, and “a (H₂O/MeOH)” are provided in the column of the “membrane permeability”. In the simulation program of the present embodiment, calculation and analysis (various graph displays (FIG. 19)) are performed for a step of synthesizing methanol (MeOH) from carbon dioxide (CO₂) and hydrogen (H₂). The variation factors that can input the parameters are “H₂”, “MeOH”, “H₂O”, “CO”, “CO₂”, and “N₂”.

When a parameter is input to “H₂”, “MeOH”, “H₂O”, “CO”, “CO₂”, and “N₂” in the input cell 1 column 52, values of some other variation factors (“a (H₂O/H₂)”, “a (H₂O/CO₂)”, and the like) are automatically calculated and displayed in the corresponding reflection cell.

In the example of FIG. 9, values of “1.00E−08” are input to “H₂”, “CO”, “CO₂”, and “N₂”, and values of “1.00E−06” are input to “MeOH” and “H₂O”. Therefore, the calculation results of “a (H₂O/H₂)”, “a (H₂O/CO₂)”, and “a (H₂O/CO)” are all “100”. In addition, the calculation result of “a (H₂O/MeOH)” is “1”.

Here, in the present embodiment, a numerical value may be expressed using “E” representing a power. For example, “1.00E−08” and “1.00E−06” mean 1×10⁻⁸ or 1×10⁻⁶. “1.0E+10” described later means 1.0×10¹⁰, and “3.00E+10⁰” means 3.00×10⁰. Furthermore, for example, “1.E+10” has the same meaning as “1.0E+10” or “1.00E+10”.

### <<Input Cell 2 Column 54>>

FIG. 10 is an enlarged view of input cell 2 column 54 in FIG. 8. In the input cell 2 column 54, a column of “pressure” and a column of “reaction separation temperature” are provided. Among these, in the column of “pressure”, columns of “supply side” and “permeation side” which are variation factors are provided. In the input cell 2 column 54, any of the “supply side” and the “permeation side” of the “pressure” and the “reaction separation temperature” is the variation factor that can input the parameter.

### <<Input Cell 3 Column 56>>

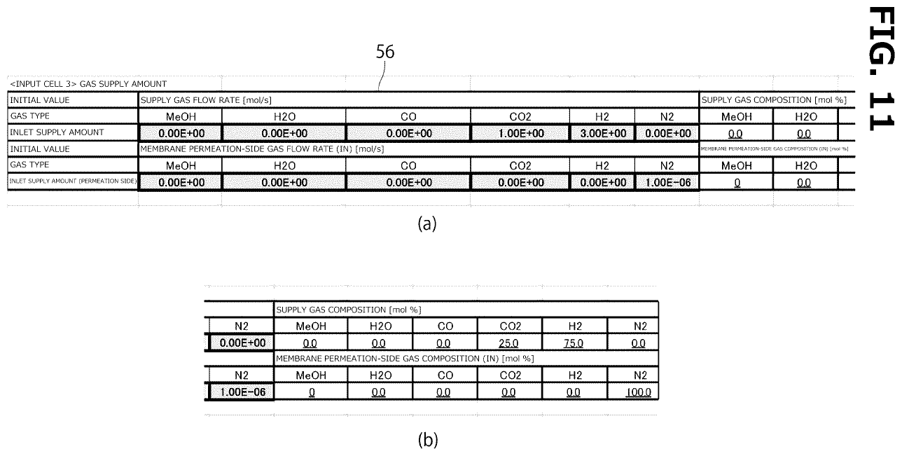

FIGS. 11(a) and 11(b) illustrate the input cell 3 column 56 of FIG. 8 in an enlarged manner. FIG. 11(a) illustrates the left side of the input cell 3 column 56, and FIG. 11(b) illustrates the right side following FIG. 11(a). In the input cell 3 column 56, an input related to a “gas supply amount” of the gas (supply gas) supplied to the membrane reactor and a calculation result based on the input value are displayed.

The left side (FIG. 11(a)) of the input cell 3 column 56 is a column of a variation factor related to a flow rate of the gas, and the right side (FIG. 11(b)) of the input cell 3 column 56 is a column of a variation factor related to a composition of the gas. On the left side (FIG. 11(a)) and the right side (FIG. 11(b)) of the input cell 3 column 56, the column of the variation factor related to the supply gas and the column of the variation factor related to the membrane permeation-side gas (gas that has permeated the membrane) are provided vertically.

Furthermore, on the left side (FIG. 11(a)) of the input cell 3 column 56, a column of a “supply gas flow rate” and a column of a “membrane permeation-side gas flow rate (IN)” are provided. On the right side (FIG. 11(b)) of the input cell 3 column 56, a column of a “supply gas composition” and a column of a “membrane permeation-side gas composition (IN)” are provided.

In the examples of FIGS. 11(a) and (b), columns of “MeOH”, “H₂O”, “CO”, “CO₂”, “H₂”, and “N₂” are provided as columns of a “gas type” related to the supply gas and the membrane permeation-side gas. For the “supply gas flow rate” and the “membrane permeation-side gas flow rate (IN)” on the left side (FIG. 11(a)) of the input cell 3 column 56, the user can input values of parameters related to an “inlet supply amount” and an “inlet supply amount (permeation side)” for each “gas type”.

When a value of the parameter is input for each “gas type”, a ratio of each gas type is automatically calculated. Then, the calculation result is automatically displayed in the reflection cell of the “inlet supply amount” or the “inlet supply amount (permeation side)” for each gas type of the “supply gas composition” and the “membrane permeation-side gas composition (IN)” on the right side (FIG. 11(b)) of the input cell 3 column 56.

In the example of FIG. 11, the “supply gas flow rate” (unit: [mol/s (sec)]) of “CO₂” on the left side (FIG. 11(a)) is “1.00E+00”, and the “supply gas flow rate” of “H₂” is “3.00E+00”. The “supply gas flow rate” for the other gas types is “0.00E+00”. Therefore, the “membrane permeation-side gas composition (IN)” (unit: [mol/%]) of “CO₂” on the right side (FIG. 11(b)) of the input cell 3 column 56 is “25”, and the “membrane permeation-side gas composition (IN)” of “H₂” is “75”. The “membrane permeation-side gas composition” for the other gas types was “0.0”.

In the example of FIG. 11, a “membrane-permeating gas flow rate (IN)” (unit: [mol/s]) of “N₂” on the left side (FIG. 11(a)) is “1.00E−06”, and the “membrane-permeating gas flow rate (IN)” related to the other gas types is “0.00E+00”. Therefore, the “membrane permeation-side gas composition (IN)” (unit: [mol/%]) of “N₂” on the right side (FIG. 11(b)) of the input cell 3 column 56 is “100.0”, and the “membrane permeation-side gas composition (IN)” related to the other gas types is “0.0”.

### <<Result Display Cell 1 Column 58>>

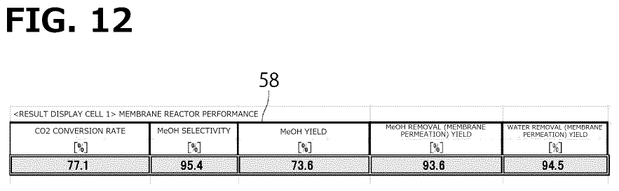

FIG. 12 illustrates the result display cell 1 column 58 in FIG. 8 in an enlarged manner. In the result display cell 1 column 58, columns of a “CO₂ conversion rate”, a “MeOH selectivity”, a “MeOH yield”, a “MeOH removal (membrane permeation) yield”, and a “water removal (membrane permeation) yield” are provided side by side in a row direction. In the result display cell 1 column 58, calculation results of a “CO₂ conversion rate”, “MeOH selectivity”, a “MeOH yield”, a “MeOH removal (membrane permeation) yield”, and a “water removal (membrane permeation) yield” are displayed according to the parameters input in the input cell 1 column 52 (FIG. 9), the input cell 2 column 54 (FIG. 10), and the input cell 3 column 56 (FIGS. 11(a) and (b)). A mathematical formula used for the calculation will be described later.

### <<Result Display Cell 2 Column 60>>

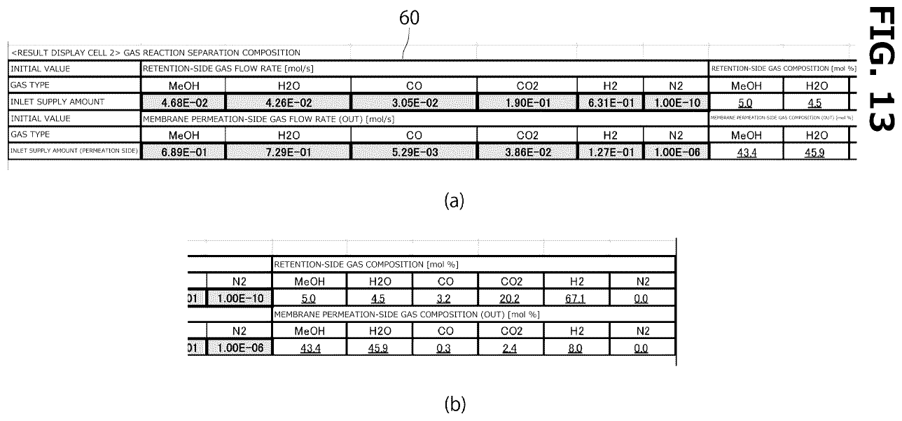

FIGS. 13(a) and 13(b) illustrate the result display cell 2 column 60 in FIG. 8 in an enlarged manner. FIG. 13(a) illustrates the left side of the result display cell 2 column 60, and FIG. 13(b) illustrates the right side following FIG. 13(a). A result display cell 2 column 60 displays the calculation result related to “gas reaction separation composition” in the membrane reactor.

The left side (FIG. 13(a)) of the result display cell 2 column 60 is the column of the variation factor related to the gas flow rate, and the right side (FIG. 13(b)) of the result display cell 2 column 60 is the column of the variation factor related to the gas composition. On the left side (FIG. 13(a)) and the right side (FIG. 13(b)) of the result display cell 2 column 60, the column of variation factors related to a retention-side gas and the column of variation factors related to the membrane permeation-side gas (gas that has permeated the membrane) are provided vertically.

Specifically, on the left side of the result display cell 2 column 60 (FIG. 13(a)), a column of a “retention-side gas flow rate” and a column of a “membrane permeation-side gas flow rate (OUT)” are provided. On the right side of the result display cell 2 column 60 (FIG. 13(b)), a column for a “retention-side gas composition” and a column of a “membrane permeation-side gas composition (OUT)” are provided.

In the example of FIG. 13, the “gas type” related to the retention-side gas and the membrane permeation-side gas is “MeOH”, “H₂O”, “CO”, “CO₂”, “H₂”, and “N₂”, similarly to the input cell 3 column 56 (FIGS. 11(a) and (b)). For each “gas type”, when the calculation result of the “inlet supply amount” or the “inlet supply amount (permeation side)” is displayed, the calculation result is automatically displayed in the reflection cell of the “inlet supply amount” or the “inlet supply amount (permeation side)” for each gas type of the “retention-side gas composition” or the “membrane permeation-side gas composition (OUT)” on the right side (FIG. 13(b)) of the result display cell 2 column 60.

### <Calculation Step S3>

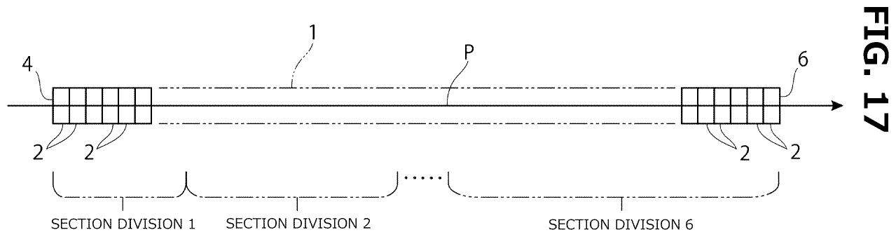

In the following [Mathematical Formula 1], a theoretical formula used for flow analysis related to a membrane reactor of an isothermal system is illustrated together with the unit cell 2. The unit cell 2 is similar to that illustrated in FIG. 1. The unit cell 2 is obtained by subdividing a series of reaction separation process steps P between an inlet 4 and an outlet 6 as illustrated in FIG. 17.

The theoretical formulas as illustrated in [Mathematical Formula 1] and [Mathematical Formula 2] are applied to each unit cell 2. In the formula of “Fⱼ₊₁=Fⱼ+EvᵢRᵢⱼW−ΣJᵢⱼ” in [Mathematical Formula 1], “ΣvᵢRᵢⱼW” is a term of chemical reaction, and “ΣJᵢⱼ” is a term of membrane permeation separation. Similarly, “ΣJᵢⱼ” in the formula of “Qⱼ₊₁=Qⱼ+ΣJᵢⱼ” in [Mathematical Formula 1] is a term of the membrane permeation separation.

In the present embodiment, a reaction rate equation described in the following literature is used.
(1) G. H. Graaf et al, Kinetics of low-pressure methanol synthesis, Chem. Eng. Sci. 43 (1988) 3185-3195.
(2) G. H. Graaf et al, Chemical Equilibria in methanol synthesis including the water-gas shift reaction: a critical reassessment, Ind. Eng. Chem. Res. 55 (2016) 5854-5864.

In these documents, theoretical formulas as illustrated in the following [Mathematical Formula 2] are also described.

A: CO+2H₂⇔CH₃OH Formula 3

B: CO₂+H₂⇔CO+H₂O Formula 4

C: CO₂+3H₂⇔CH₃OH+H₂O Formula 5

$${r}_{{CH}_{3} OH , A 3}^{′} = \frac{{k}_{p s , A 3}^{′} {K}_{CO} [ {f}_{CO} {f}_{{H}_{2}}^{3 / 2} - {f}_{{CH}_{3} OH} / ( {f}_{{H}_{2}}^{1 / 2} {K}_{{p}_{1}}^{o} ) ]}{( 1 + {K}_{CO} {f}_{CO} + {K}_{{CO}_{2}} {f}_{{CO}_{2}} ) [ {f}_{{H}_{s}}^{1 / 2} + ( {K}_{{H}_{2} O} / {k}_{{H}_{2}}^{1 / 2} ) {f}_{{H}_{2} O} ]} \qquad Formula 6$$

$${r}_{{H}_{2} O , B 2}^{′} = \frac{{k}_{p s , B 2}^{′} {K}_{{CO}_{2}} [ {f}_{{CO}_{2}} {f}_{{H}_{2}} - {f}_{{H}_{2} O} {f}_{CO} / {K}_{{p}_{2}}^{o} )}{( 1 + {K}_{CO} {f}_{CO} + {K}_{{CO}_{2}} {f}_{{CO}_{2}} ) [ {f}_{{H}_{s}}^{1 / 2} + ( {K}_{{H}_{2} O} / {k}_{{H}_{2}}^{1 / 2} ) {f}_{{H}_{2} O} ]} \qquad Formula 7$$

$${r}_{{CH}_{3} OH , C 3}^{′} = \frac{{k}_{p s , C 3}^{′} {K}_{{CO}_{3}} [ {f}_{{CO}_{2}} {f}_{{H}_{2}}^{3 / 2} - {f}_{{CH}_{3} OH} {f}_{{H}_{2} O} / ( {f}_{{H}_{2}}^{3 / 2} {K}_{{p}_{3}}^{o} ) ]}{( 1 + {K}_{CO} {f}_{CO} + {K}_{{CO}_{2}} {f}_{{CO}_{2}} ) [ {f}_{{H}_{s}}^{1 / 2} + ( {K}_{{H}_{2} O} / {k}_{{H}_{2}}^{1 / 2} ) {f}_{{H}_{2} O} ]} \qquad Formula 8$$

$$( = {r}_{{H}_{2} O , C 3}^{′} )$$

$${k}_{p s , A 3}^{′} = ( 2.69 \pm 0.14 ) \times {10}^{7} \times exp ( \frac{- 109 , 900 \pm 200}{RT} ) \qquad Formula 9$$

$${k}_{p s , B 2}^{′} = ( 7.31 \pm 4.9 ) \times {10}^{8} \times exp ( \frac{- 123 , 400 \pm 1600}{RT} ) \qquad Formula 10$$

$${k}_{p s , C 3}^{′} = ( 4.36 \pm 0.25 ) \times {10}^{8} \times exp ( \frac{- 65 , 200 \pm 200}{RT} ) \qquad Formula 11$$

$${K}_{CO} = ( 7.99 \pm 1.28 ) \times {10}^{- 7} \times exp ( \frac{38 , 100 \pm 600}{RT} ) \qquad Formula 12$$

$${K}_{{CO}_{2}} = ( 1.02 \pm 0.16 ) \times {10}^{- 7} \times exp ( \frac{67 , 400 \pm 600}{RT} ) \qquad Formula 13$$

$${K}_{{H}_{2} O} / {K}_{{H}_{2}}^{1 / 2} = ( 4.13 \pm 1.51 ) \times {10}^{- 11} \times exp ( \frac{104 , 500 \pm 1100}{RT} ) . \qquad Formula 14$$

$$ln {Kp}_{1} ° ( T ) = \frac{1}{RT} [ {a}_{1} + {a}_{2} T + {a}_{3} {T}^{2} + {a}_{4} {T}^{3} + {a}_{5} {T}^{4} + {a}_{6} {T}^{5} + {a}_{7} T ln T ] \qquad Formula 15$$

$${a}_{1} = 7.4414 \times {10}^{+ 4} ; {a}_{2} = 1.8926 \times {10}^{+ 2} ; {a}_{3} = 3.2443 \times {10}^{- 2} ; {a}_{4} = 7.0432 \times {10}^{- 6} ; {a}_{5} = - 5.6053 \times {10}^{- 9} ; {a}_{6} = 1.0344 \times {10}^{- 12} ; {a}_{7} = - 6.4364 \times {10}^{+ 1} .$$

$$ln {Kp}_{2} ° ( T ) = \frac{1}{RT} [ {b}_{1} + {b}_{2} T + {b}_{3} {T}^{2} + {b}_{4} {T}^{3} + {b}_{5} {T}^{4} + {b}_{6} {T}^{5} + {b}_{7} T ln T ] \qquad Formula 16$$

$${b}_{1} = - 3.94121 \times {10}^{+ 4} ; {b}_{2} = - 5.41516 \times {10}^{+ 1} ; {b}_{3} = - 5.5642 \times {10}^{- 2} ; {b}_{4} = 2.576 \times {10}^{- 5} ; {b}_{5} = - 7.6594 \times {10}^{- 9} ; {b}_{6} = 1.0161 \times {10}^{- 12} ; {b}_{7} = 1.8429 \times {10}^{+ 1} .$$

$${Kp}_{3} ° = {Kp}_{1} ° {Kp}_{2} ° \qquad Formula 17$$

As described above, the membrane reactor 1 according to the present embodiment integrates the chemical reaction and the membrane separation to separate the separation symmetric substance from the supply fluid. The simulation program used in the calculation step S3 includes a portion describing the theoretical formula and the variable described in the above literatures.

In other words, at least a part of the simulation program used in the present embodiment is described using the extended function language (in this case, VBA) for giving the extended function to the application program with the calculation function (in this case, spreadsheet software) installed in the client device 30 (user-side terminal device), and causes the user-side terminal device to analyze the flow step of the membrane reactor in which a chemical reaction and membrane separation are integrated by a predetermined development support method (here, the development support method includes the input step S2 to the analysis result display step S4.).

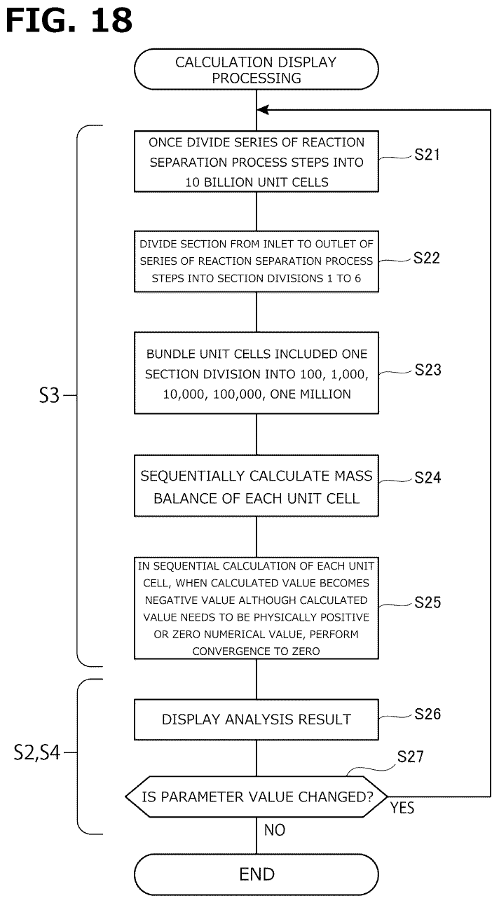

FIG. 18 schematically illustrates a flow of main processing (here, referred to as “calculation display processing”) performed in the client device 30 from the calculation step S3 to the analysis result display step S4. The processing of S21 to S25 in FIG. 18 is performed in the calculation step S3, and the subsequent processing of S26 is performed in the input step S2 or the analysis result display step S4 described later.

Here, in FIG. 18, for convenience of description, S24 (sequential calculation processing) and S25 (processing of converging the calculated value) are described to be divided, and the processing of S25 is described to be performed after S24. However, in the present embodiment, as described later, S24 (sequential calculation processing) is repeated for each unit cell 2. Therefore, strictly speaking, S25 (processing of converging the calculated value) can be considered to be incorporated into S24.

The calculation step S3 includes the step (such as S21 in FIG. 18) of once dividing the series of reaction separation process steps into the first predetermined number (such as 10,000 to ten billion) of unit cells (such as the unit cells 2), the step (such as S22 in FIG. 18) of dividing the section from the inlet (such as the inlet 4 in FIG. 17) to the outlet (such as the outlet 6 in FIG. 17) of the series of reaction separation process steps into the plurality of sections (such as sections of section divisions 1 to 6 described later) that can be represented by the number of unit cells, the step (such as S23 in FIG. 18) of bundling the unit cells included in at least one section into the second predetermined number (1,000, 90, 90, 90, 90, 9,990, or the like) smaller than the first predetermined number, and the step (such as S24 in FIG. 18) of calculating at least one of a mass balance and an energy balance of each unit cell from the inlet to the outlet of the series of reaction separation process steps by sequential calculation.

As described above, by bundling the unit cells included in the divided sections into the second predetermined number smaller than the first predetermined number, the behavior of each stage of the series of reaction separation process steps can be clarified. That is, not only the outlet 6 of the series of reaction separation process steps but also each partial step between the inlet 4 and the outlet 6 can be analyzed. Then, it is possible to create information that can be used for visualizing each partial step in the analysis result display step S4 described later.

In the sequential calculation, an operation of solving a formula relating to at least one of the mass balance and the energy balance in each unit cell, determining an input of the next (next stage) cell based on the solution, and similarly obtaining a solution in the next (next stage) unit cell is sequentially repeated. For this reason, for example, when the sequential calculation is performed first for each of the first predetermined number (10,000 to ten billion or the like) of unit cells, a lot of time is spent. For this purpose, the unit cells are divided and bundled, and the cells are enlarged. By such an operation, the number of cells to be calculated is reduced to save time.

According to such a development support system 10, since the behavior of each stage of the series of reaction separation process steps can be clarified by a simple method, for example, many users who have not received special training for using a simulation program can easily perform the simulation related to a membrane reactor.

Here, the relationship between the first predetermined number and the second predetermined number only needs to be such that the number of unit cells 2 bundled in each section (such as section of section divisions 1 to 6 described later) is smaller than the first predetermined number that is the number of unit cells 2. For example, when the first predetermined number is ten billion, the second predetermined number may include 100,000, one million, or the like.

In the following [Mathematical Formula 3], the calculation content (numerical model) of flow analysis (simulation for a non-isothermal system membrane reactor) related to a non-isothermal system membrane reactor is illustrated together with a physical model.

The above [Mathematical Formula 1] relates to the sequential calculation of the mass balance (isothermal system), and this [Mathematical Formula 3] relates to the sequential calculation of the energy balance addition (non-isothermal system). The examples in FIGS. 8 to 13 are examples in a case where sequential calculation of [Mathematical Formula 1] (S24 in FIG. 18) is performed.

Note that, in the calculation step S3, only one type of analysis among a plurality of types of analysis as illustrated in [Mathematical Formula 1] and [Mathematical Formula 3] may be performed, or a plurality of types of analysis may be selected and performed.

In a case where the analysis can be selected in this manner, the selection of the analysis can be performed by the user, for example, in the input step S2 illustrated in FIG. 4. When selecting the type of analysis, for example, the user can select and open an analysis file (spreadsheet file) corresponding to the type.

Alternatively, for example, it is also possible to provide a function of a pull-down menu (not illustrated) on the parameter input screen 50 (FIG. 8) so that a list of types of analysis is displayed when the user expands the pull-down menu. In this case, when the user selects a desired type of analysis, an arithmetic formula (for example, an arithmetic formula illustrated in [Mathematical Formula 1] or [Mathematical Formula 3]) corresponding to the selected type can be used for the analysis.

The following [Table 1] illustrates an integration example of cell division related to S22 and S23 in FIG. 18. The integration example of [Table 1] illustrates an example of the relationship between the first predetermined number and the second predetermined number described above. The first predetermined number is the total number of unit cells 2 when the series of reaction separation process steps is once equally divided (FIG. 17) into the first predetermined number (10,000 to ten billion the like) of unit cells. The second predetermined number is the number of bundles (1,000, 90, 90, 90, 90, 9,990, or the like) obtained by bundling the unit cells 2 included in the plurality of sections (such as the section of the section divisions 1 to 6) from the inlet 4 to the outlet 6 of the series of reaction separation process steps. Hereinafter, this integration example will be described.

TABLE 1
Integration Example of Cell Division

| | Cell Order | | Number of |
|---|---|---|---|
| Section Division | Section Start | Section End | Integrated Cells |
| 1 | 1 | 1.E+03 | 1 |
| 2 | 1,001 | 1.E+04 | 100 |
| 3 | 10,001 | 1.E+05 | 1,000 |
| 4 | 100,001 | 1.E+06 | 10,000 |
| 5 | 1,000,001 | 1.E+07 | 100,000 |
| 6 | 10,000,001 | 1.E+10 | 1,000,000 |

In the example of [Table 1], the series of reaction separation process steps is divided into six sections of 1 to 6 as illustrated in the column of the “section division”. The series of reaction separation process steps is divided into ten billion (1.E+10) unit cells. The unit cell 2 constituting the inlet 4 (FIG. 17) of the series of reaction separation process steps is the first unit cell of the ten billion unit cells, and the unit cell constituting the outlet 6 is the last unit cell of ten billion unit cells.

The size of each section can be expressed by the number of unit cells 2. In the example of [Table 1], the section with the “section division” of “1” is constituted by 1,000 unit cells 2 of the first to 1000 (1.E+03) as illustrated in the uppermost row (the row with the section division of “1”) in the columns of a “section start” and a “section end” of a “cell order”. Hereinafter, the section in which the “section division” is “1” to the section in which the “section division” is “6” are referred to as “section division 1” to “section division 6”.

In the example of [Table 1], the section division 2 includes 9,000 unit cells 2 of 1,001 to 10,000 (1.E+04, 10,000). The section division 3 includes 90,000 unit cells 2 of 10,001 to 100,000 (1.E+05, 100,000).

The section division 4 includes 900,000 unit cells 2 of 100,001 to one million (1.E+06, one million). The section division 5 includes nine million unit cells 2 of 1,000,001 to ten million (1.E+07, ten million). The section division 6 includes 9.99 billion unit cells 2 of 10,000,001 to ten billion (1.E+10).

In the example of [Table 1], the “number of integrated cells” means the number of unit cells 2 integrated by being bundled. The “number of integrated cells” in the section division 1 is one. Therefore, in the example of [Table 1], 1,000 bundles are formed in the section division 1. Here, in a case where the “integrated cell number” is one, it can be considered that it does not correspond to “being bundled” or “bundling”. However, here, even in a case where the “number of integrated cells” is one, it is assumed that each integrated cell corresponds to “being bundled” or “bundling”. Note that the present invention is not limited to this, in a case where only one unit cell 2 remains, it is does not correspond to “being bundled” or “bundling”, but in a case where the plurality of unit cells 2 are integrated, it can correspond to “being bundled” or “bundling”.

The “number of integrated cells” in the section divisions 2 to 6 is 100, 1,000, 10,000, 100,000, and one million, respectively. Among them, the section division 2 includes 9,000 unit cells. Therefore, 90 (=9,000/100) bundles are formed in the section division 2.

$$Similarly , 90 ( = 90 , 000 / 1 , 000 , = 900 , 000 / 10 , 000 , = nine million / 100 , 000 )$$

bundles are formed for the section divisions 3 to 5. Since the section division 6 includes 9.99 billion unit cells, 9,990 (=9.99 billion/one million) bundles are formed.

In the section divisions 1 to 6, the number of integrated cells increases from the section division 1 including the inlet 4 to the section division 6 including the outlet 6 illustrated in FIG. 17. For the bundle of integrated unit cells 2, constants described in the simulation program are common for each bundle. Specifically, when the simulation program is created on the PC, the work of copying and pasting of the same description is repeated for each bundle, and the code is described. For example, in the case of the section division (here, the section divisions 2 to 5) in which the number of bundles is 90, copying and pasting of the same description are performed 89 (=90−1) times.

In this manner, performing the same description for each bundle can be referred to as, for example, “unit cell grouping” or “description grouping”. Hereinafter, these are collectively referred to as the “grouping”. Such “grouping” is performed in consideration of the tendency of the reaction separation process step.

In the series of reaction separation process steps, a change amount in the numerical value of the “membrane reactor characteristic” tends to be relatively large on a side (a side where the numerical value of the “membrane effective length direction” is small) close to the inlet 4 of the reaction separation process step, and a change amount in the numerical value of the “membrane reactor characteristic” tends to be relatively small on a side (a side where the numerical value of the “membrane effective length direction” is large) of the outlet 6. That is, in the reaction separation process step, the change amount in the numerical value for each unit cell 2 tends to be smaller on the outlet 6 side than on the inlet 4 side.

For this reason, the description of the simulation program can be facilitated by performing the “grouping” as described above for the series of reaction separation process steps. Furthermore, the same processing is repeated on the spreadsheet software by the “grouping”, and the processing of the client device 30 can be simplified. As a result, the simulation of the series of reaction separation process steps can be quickly performed in a short time.

Note that, for the grouping section (here, each of the section divisions 1 to 6), knowledge (or a result of trial and error) of a supplier providing the application program is used to determine the unit cells of the start (head) and the end (tail). Then, adjustment is made according to the application and purpose of the membrane reactor 1 so as not to cause an abnormality such as divergence in the calculation result. In addition, the “grouping” is performed in consideration of performance of a PC (client device 30) assumed to be used for simulation.

### <Program Non-Display Processing>

Here, in the present embodiment, processing (program non-display processing) of non-displaying the content of the simulation program so that the user cannot view the content of the simulation program is performed on the simulation program described in an extended function language (in this case, VBA). As the program non-display processing, a password for code display can be set. The password for code display is set in the service provider, and the password is not disclosed to the user.

The program non-display processing can be realized by using a password setting function provided in an application (editor) for editing an extended function language (in this case, the VBA). The content of the simulation program can be converted into a black box by performing the program non-display processing. Then, it is possible to prevent the simulation program from being imitated and more reliably recover the investment related to the program development.

### <Analysis Result Display Step S4>

The calculation result (FIG. 12, FIG. 13) using the input values to the input cell 1 column 52, the input cell 2 column 54, and the input cell 3 column 56 illustrated in FIGS. 9 to 11 and a process in the middle of the calculation are used to display an analysis result (analysis result display) as illustrated in FIG. 19. FIG. 19 illustrates an example of the analysis result display screen 80. The display of the analysis result display screen 80 corresponds to the step of S26 in FIG. 18.

On the analysis result display screen 80, a plurality of graphs are displayed side by side on the same work sheet of spreadsheet software. Each graph is created using the graph creation function of the spreadsheet software and using the input parameter value, calculated value, and calculation result.

In the example of FIG. 19, 12 graphs are displayed side by side in a matrix in a horizontal direction and a vertical direction. Three graphs are arranged in the horizontal direction, and four graphs are arranged in the vertical direction. Hereinafter, the position of each graph is represented by the position (order) in the row direction (horizontal direction) and the column direction (vertical direction) starting from the left end of the uppermost row as necessary.

The 12 graphs in the example of FIG. 19 are classified into a graph relating to “membrane reactor performance”, a graph relating to “membrane retaining-side characteristics”, and a graph relating to “membrane permeation-side characteristics”. The three graphs (the graphs of the first row and the first column to the first row and the third column) in the uppermost row are graphs relating to “membrane reactor performance”, and in particular, graphs indicating “basic characteristics” of the membrane reactor are displayed side by side in the uppermost row.

The six graphs in the second row and the first column and the second column to the fourth row and the first column and the second column are graphs relating to the “membrane retaining-side characteristics”. Among the graphs relating to the “membrane retaining-side characteristics”, three graphs on the left half (from the second row and the first column to the fourth row and the first column) are graphs representing the entire “membrane retaining-side characteristics”, and three graphs on the right half (from the second row and the second column to the fourth row and the second column) are graphs in which a part of a vertical axis of the graph representing the entire left is enlarged. Three graphs in the second row and the third column to the fourth row and the third column are graphs representing all or a part (excerpt) of the “membrane permeation-side characteristics”.

The horizontal axes of the 12 graphs in the example of FIG. 19 are common, and the distance in the membrane effective length direction is indicated in the range of 0 to 150 [m]. Among the three graphs relating to “basic characteristics” in the uppermost row, the vertical axis of each of the graphs of the first row and the first column to the first row and the third column represents the “CO₂ conversion rate”, “MeOH reaction conversion rate”, and “MeOH yield”.

The graphs in the second row and the first column and the second row and the second column illustrate the “gas flow rate” among the six graphs in the second row and the first column and the second column to the fourth row and the first column and the second column regarding “membrane retaining-side characteristics”. The graphs in the third row and the first column and the third row and the second column indicate the “partial pressure”, and the graphs in the fourth row and the first column and the fourth row and the second column indicate the “concentration”.

The vertical axis of the graph in the second row and the first column indicates the “membrane retaining-side gas flow rate” in the range of 5.0E+00 to 3.5E+00 [mol/s], and the vertical axis of the graph in the second row and the second column indicates the range of 5.0E+00 to 2.5E−00 [mol/s] in the “membrane retaining-side gas flow rate” in an enlarged manner.

The vertical axis of the graph in the third row and the first column indicates the “membrane retaining-side partial pressure” in the range of 0 to 40 [bar], and the vertical axis of the graph in the third row and the second column indicates the range of 0.00 to 3.0 [bar] in the “membrane retaining-side partial pressure” in an enlarged manner.

The vertical axis of the graph in the fourth row and the first column indicates the “membrane retaining-side gas concentration” in the range of 0.0 to 80.0 [%], and the vertical axis of the graph in the fourth row and the second column indicates the range of 0.0 to 6.0 [%] in the “membrane retaining-side gas concentration” in an enlarged manner.

The graph in the second row and the third column among the three graphs in the second row and the third column to the fourth row and the third column regarding the “membrane permeation-side characteristics” indicates the “gas flow rate”. The graph in the third row and the third column indicates the “partial pressure”, and the graph in the fourth row and the third column indicates the “concentration”.

The vertical axis of the graph in the second row and the third column indicates the “membrane permeation-side gas flow rate” in the range of 0.0E+00 to 8.0E−01 [mol/s], and the vertical axis of the graph in the third row and the third column indicates the “membrane permeation-side partial pressure” in the range of 0.00 to 0.60 [bar]. The vertical axis of the graph in the fourth row and the third column indicates the “membrane permeation-side gas concentration” in the range of 0.0 to 120.0 [%].

After the analysis result is displayed, the user can individually change the parameter value with respect to the variation factors of the predetermined membrane reactor characteristics and the predetermined reaction separation conditions. This change in the parameter value corresponds to the step of S27 in FIG. 18. When the parameter value is changed in S27 (S27: YES), the process returns to S21, and the calculation step (S21 to S25) is executed again. When there is no change in the parameter value in S27 (S27: NO), the calculation display processing ends.

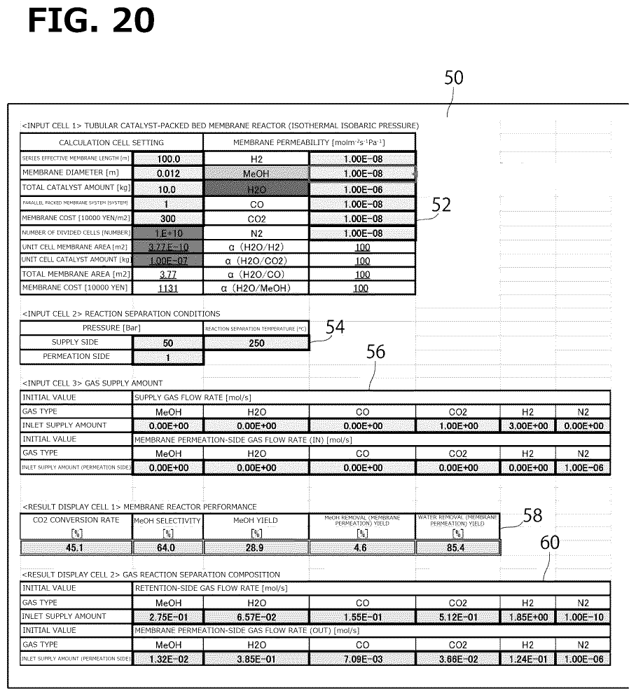

FIG. 20 illustrates a change of a variation factor when “1.00E−06” of “MeOH” of “membrane permeability” in input cell 1 column 52 illustrated in FIGS. 8 and 9 is changed to “1.00E−08”. In FIG. 20, a rectangular frame line is superimposed on the input cell of “MeOH”. By changing the parameter value of “MeOH”, the value of “a (H₂O/MeOH)” of “membrane permeability” in the same input cell 1 column 52 is changed from “1” to “100”.

Further, when FIG. 20 is compared with the result display cell 1 column 58 in FIG. 12, the “CO₂ conversion rate”, “MeOH selectivity”, “MeOH yield”, “MeOH removal (membrane permeation) yield”, and “water removal (membrane permeation) yield” are changed from “77.1”, “95.4”, “73.6”, “93.6”, and “94.5” to “45.1”, “64.0”, “28.9”, “4.6”, and “85.4” as illustrated in FIG. 20.

Similarly, in the result display cell 2 column 60, each variation factor changes the parameter value, and thus, as illustrated in FIGS. 13 and 20, values other than “N₂” of the “retention-side gas flow rate” and “membrane permeation-side gas flow rate (OUT)” are changed. Here, FIG. 13 illustrates the column of the “retention-side gas composition” and the column of the “membrane permeation-side gas composition (OUT)” on the right side of the result display cell 2 column 60, but these columns are not illustrated in FIG. 20.

As described above, the influence exerted on the analysis result by the individual change in each of the variation factors such as the parameter related to the membrane reactor characteristic (FIG. 9) and the parameter related to the reaction separation conditions (FIG. 10) is individually reflected, and the user can individually grasp the influence when each variation factor is changed. As a result, it is possible to achieve an effect that optimization of each variation factor such as the membrane reactor characteristics and the reaction separation conditions can be efficiently advanced.

By graphically displaying the analysis result in the analysis result display step S4, each partial step can be visualized. The behavior of each stage of the series of reaction separation process steps can be clearly illustrated by plotting the calculation result not only for the outlet 6 (FIG. 17) but also for each partial step in the middle.

When the parameter value is changed, the analysis result is immediately displayed for each changed parameter value. Therefore, the user can quickly grasp the analysis result when each parameter value is changed, including the intermediate stage of the series of reaction separation process steps. This also makes it possible to achieve an effect that optimization of each variation factor of membrane reactor characteristics and reaction separation conditions can be efficiently advanced.

### <Divergence Suppression Processing in Calculation Step S3>

In the calculation step S3 described above, the divergence suppression processing for suppressing the divergence of the calculation result is performed. The divergence suppression processing corresponds to the step of S25 in FIG. 18. The divergence suppression processing is the processing for converging the calculated value by replacing the calculated value with another value when the calculated value diverges in the sequential calculation (more precisely, sequential calculation relating to unit cells (integrated cells) enlarged by bundling) related to each unit cell 2.

More specifically, in the divergence suppression processing, when the calculated value becomes a negative value although the calculated value needs to be physically a positive or zero numerical value in the sequential calculation related to each unit cell (integrated cell), the value is converged to one of the same value as the immediately preceding unit cell (integrated cell), zero, and a value that is infinitesimally small unless being zero.

In the present embodiment, convergence to zero is performed in the divergence suppression processing. However, the present invention is not limited thereto, and in the divergence suppression processing, convergence to the same value as that of the immediately preceding unit cell or convergence to a small value as long as the value is not zero may be performed.

By providing the step of the divergence suppression processing in the calculation step S3, the problem of abnormal divergence of numerical values can be suppressed. That is, in general, when a certain same range is divided into cells, if the cells are divided into large cells, the change amount that can be followed is small (the allowable change amount in the range is small), and if the cells are divided into small cells, the change amount that can be followed is large (the allowable change amount in the range is large). Therefore, the smaller the number of unit cells to be divided, the smaller the allowable change amount, and when the change amount exceeds the allowable change amount, the numerical value loses physical significance and the numerical value (numerical value of the calculation result) diverges, and a normal analysis value cannot be obtained.

In other words, when the “number of divided cells” (FIG. 8) is constant, the size of the unit cell 2 changes according to the input value of the “series effective membrane length”. As the value of the “series effective membrane length” increases, the size of each unit cell 2 (the size in the direction in which the reaction separation process step proceeds) increases. With the same “series effective membrane length”, each unit cell 2 becomes larger and the allowable change amount becomes smaller as the number of unit cells is smaller. Furthermore, when the change amount allowed in the unit cell 2 exceeds a predetermined amount, the numerical value diverges and becomes a negative value without converging, and as a result, a normal analysis value cannot be obtained.

Such divergence is more likely to occur by “grouping” the series of reaction separation process steps as described above. In addition, it is not preferable that the unit cell 2 is too large in order to obtain the simulation result as accurate as possible. Furthermore, in the case of the membrane reactor, since the reaction is promoted between the inlet 4 and the outlet 6 of the series of reaction separation process steps, there is also a circumstance that divergence is likely to occur.

Under these circumstances, when the calculated value becomes a negative value, the problem of abnormal divergence of the numerical value can be suppressed by converging the numerical value to a constant value. Even when divergence occurs in any of the unit cells 2, it is possible to display the analysis result while maintaining the overall tendency.

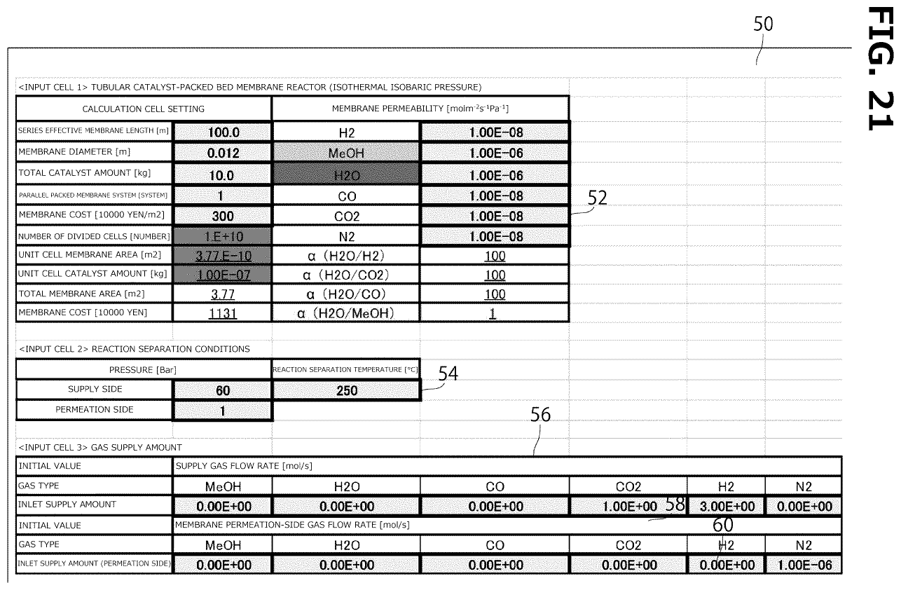

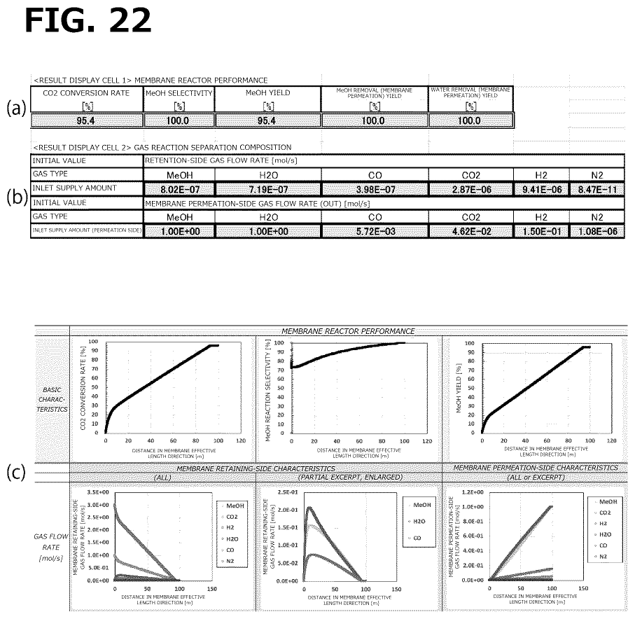

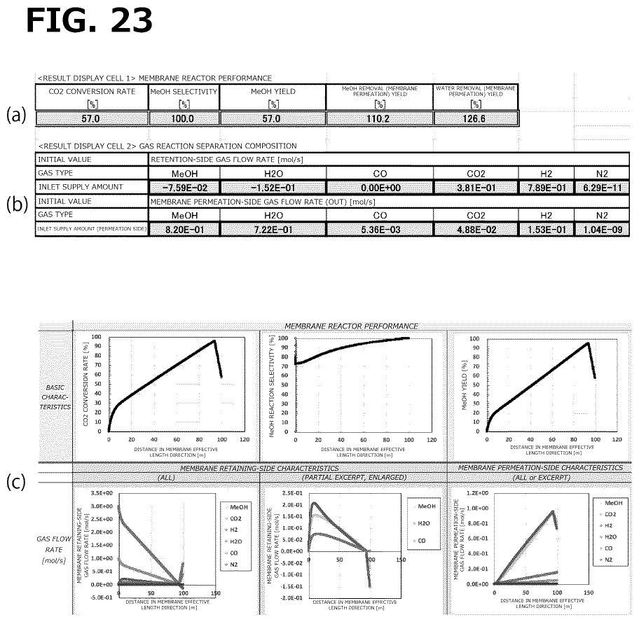

FIGS. 21 and 22(a) to 22(c) illustrate examples of input values (FIG. 21), calculation results (FIGS. 22(a) and (b)), and analysis results (FIG. 22(c)) when the simulation program of the present embodiment is used. Meanwhile, FIGS. 23(a) to 23(c) illustrate the calculation result (FIG. 23(a)) and the analysis result (FIGS. 23(b) and (c)) in a case where simulation is performed using the same input value (FIG. 21) without using the divergence suppression processing.

When the case with the divergence suppression processing in FIGS. 22(a) and (b) is compared with the case without the divergence suppression processing in FIGS. 23(a) and (b), the “CO₂ conversion rate”, the “MeOH selectivity”, the “MeOH yield”, the “MeOH removal (membrane permeation) yield”, and the “water removal (membrane permeation) yield” related to the result display cell 1 column 58 in FIG. 22(a) are “95.4”, “100.0”, “95.4”, “100.0”, and “100.0”, respectively.

“MeOH”, “H₂O”, “CO”, “CO₂”, “H₂”, and “N₂” of the “retention-side gas flow rate” of related to the result display cell 2 column 60 in FIG. 22(b) are “8.02E−07”, “7.19E−07”, “3.98E−07”, “2.87E−07”, “9.41E−07”, and “8.47E−07”, respectively.

Similarly, “MeOH”, “H₂O”, “CO”, “CO₂”, “H₂”, and “N₂” of the “membrane permeation-side gas flow rate (OUT)” related to the result display cell 2 column 60 in FIG. 22(b) are “1.00E+00”, “1.00E+00”, “5.72E−03”, “4.62E−02”, “1.50E−01”, and “1.08E−09”, respectively.

Meanwhile, in FIG. 23(a), the “CO₂ conversion rate”, “MeOH selectivity”, “MeOH yield”, “MeOH removal (membrane permeation) yield”, and “water removal (membrane permeation) yield” related to the result display cell 1 column 58 are “57.0”, “100.0”, “57.0”, “110.2”, and “126.6”, respectively, even when the input value (FIG. 21) is the same.

“MeOH”, “H₂O”, “CO”, “CO₂”, “H₂”, and “N₂” of the “retention-side gas flow rate” related to the result display cell 2 column 60 in FIG. 23(b) are “−7.59E−02”, “−1.52E−01”, “0.00E+00”, “3.81E−01”, “7.89E−01”, and “6.29E−11”, respectively.

Similarly, “MeOH”, “H₂O”, “CO”, “CO₂”, “H₂”, and “N₂” of the “membrane permeation-side gas flow rate (OUT)” related to the result display cell 2 column 60 in FIG. 23(b) are “8.20E−01”, “7.22E−01”, “5.36E−03”, “4.88E−02”, “1.53E−01”, and “1.04E−09”, respectively.

As described above, even when the input values are the same between the case with the divergence suppression processing and the case without the divergence suppression processing, completely different calculation results are obtained.

Subsequently, the analysis results of FIGS. 22(c) and 23(c) are compared. Regarding the “CO₂ conversion rate” and the “MeOH yield” in the “basic characteristics”, comparing FIG. 22(c), which is the analysis result in the case of the presence of the divergence suppression processing, with FIG. 23(c), which is the analysis result in the case of the absence of the divergence suppression processing, in FIG. 23(c), the curves are all sharply decreased from around 95 m in the “membrane effective length direction”.

Comparing “(all)” of the “gas flow rates” in the “membrane retaining-side characteristics”, in FIG. 23(c), many curves are sharply bent in the vertical direction before 100 m in the “membrane effective length direction”. Comparing the “(partial excerpt, enlarged)” of the “gas flow rate”, in FIG. 23(c), the two curves are sharply bent downward before 100 m in the “membrane effective length direction”.

Comparing the “membrane permeation-side characteristics”, in FIG. 23(c), some curves are sharply bent downward before 100 m in the “membrane effective length direction”.

As described above, even when the input values are the same between the case with the divergence suppression processing and the case without the divergence suppression processing, greatly different analysis results are obtained from before 100 m in the “membrane effective length direction”.

### <Use of if Statement>

The divergence suppression processing is described using an if statement (also referred to as “if else syntax” or the like) in the simulation program. This if statement is one of methods for creating a logical function. The client device 30 determines whether or not the condition is satisfied by executing the if statement, and branches the processing between a case where the condition is satisfied (true case) and a case where the condition is not satisfied (false case).

Specifically, in the sequential calculation of each unit cell, when the calculated value becomes a negative value although it needs to be physically a positive or zero numerical value (when corresponding to a condition), the numerical value is rewritten to zero and convergence to zero is performed.

Such an if statement is used for sequential calculation of each unit cell (integrated cell), and for each unit cell (integrated cell), work of copying and pasting of the same description related to the if statement is repeated, and a code is described.

### <Back Calculation Step Using Goal Seek Function>

The development support system 10 of the present exemplary embodiment has a function (back calculation function) of back calculating the value of the variation factor in order to achieve a target analysis result. This back calculation function is executed using a function provided in spreadsheet software. For example, Excel (trade name) has a goal seek function. The goal seek function is a function of back calculating a numerical value for obtaining a result from a calculation result of a mathematical formula. By using the goal seek function, the inverse calculation value can be obtained using the input mathematical formula.

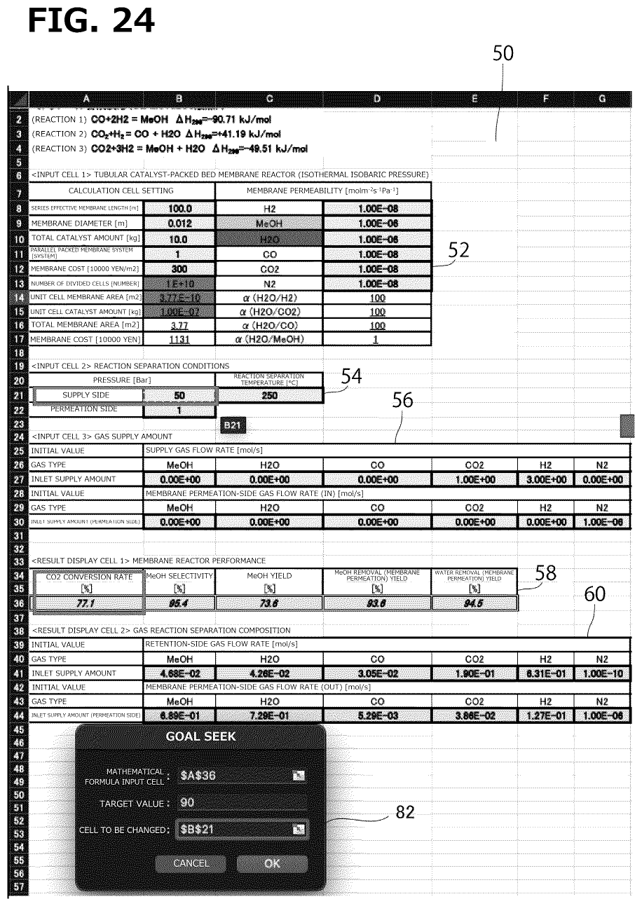

FIG. 24 illustrates a screen in a situation where the goal seek function is used for the “pressure” of the “supply side” in the input cell 2 column 54 and the “CO₂ conversion rate” in the result display cell 1 column 58. Rectangular frame lines superimposed on the input cell 2 column 54 and the result display cell 1 column 58 are superimposed later in order to clearly indicate the display positions of the “pressure” on the “supply side” and the “CO₂ conversion rate”. A value of “50” is input to the input cell corresponding to the “pressure” of the “supply side”, and the calculation result of “95.4” is displayed in the result display cell corresponding to the “CO₂ conversion rate”.

A goal seek screen 82 on which the title of the “goal seek” is displayed is superimposed and displayed on the lower left of the screen. On the goal seek screen 82, the position of the result display cell corresponding to the “CO₂ conversion rate” is input as the “mathematical formula input cell”. In addition, “90” is input as the “target value”, and the position of the input cell corresponding to the “pressure” on the “supply side” is input as the “cell to be changed”. When the user operates an “OK” button (OK button) on the goal seek screen 82, the goal seek function is executed.

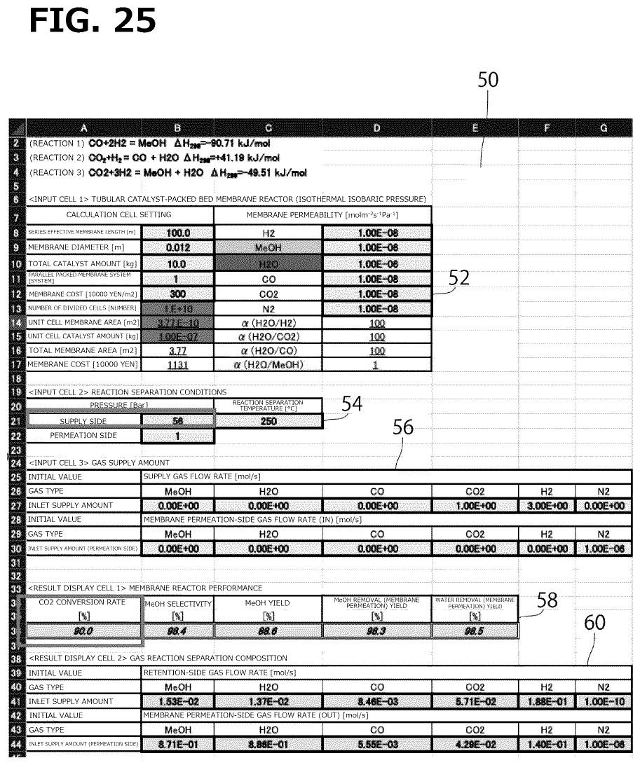

FIG. 25 illustrates a screen of a situation after the goal seek function is executed. By execution of the goal seek function, “90” of the “target value” is displayed in the result display cell corresponding to the “CO₂ conversion rate”, and the value of “56” is displayed in the input cell corresponding to the “pressure” of the “supply side”. That is, in the example of FIG. 25, it is calculated that the value of the “pressure” on the “supply side” may be set to “56” in order to set the value of “CO₂ conversion rate” to “90”.

By executing the back calculation step in this manner, the values of the respective variation factors of the membrane reactor characteristics and the reaction separation conditions necessary for achieving the target analysis result are back-calculated. Therefore, the user can quickly grasp the values of the respective variation factors of the membrane reactor characteristics and the reaction separation conditions necessary for achieving the target analysis result. As a result, it is possible to achieve an effect that the user can efficiently optimize each variation factor of the membrane reactor characteristics and the reaction separation conditions.

### Other Effects Achieved by Invention According to Present Embodiment

According to development support system 10 of the present exemplary embodiment, the unit cells included in the section divisions 1 to 6 (Table 1) are bundled into a predetermined number (1,000, 90, 90, 90, 90, 9,990). Therefore, the sequential calculation can be performed for each bundled and integrated cell (integrated cell), and the sequential calculation can be simplified. Therefore, in the client device 30 having generally limited processing capability, it is possible to perform a simulation related to the series of reaction separation process steps. Then, since the sequential calculation is simplified, it is easy to perform the simulation related to the series of reaction separation process steps using the spreadsheet software installed in the client device 30 or the extended function of the spreadsheet software.

When the inventors executed the simulations illustrated in FIGS. 8 to 13 and 19 in the client device 30 using a commercially available general-purpose PC as the client device 30, the time required for the work from the calculation step S3 to the analysis result display step S4 (until the analysis result is displayed by performing sequential calculation) was 10 seconds or less. This is 1/10 to 1/100 or less when similar simulation is performed by commercially available software for fluid numerical analysis. In a breakdown of the required time, two seconds were required for sequential calculation by the spreadsheet software, and five seconds were required for display of the graph (FIG. 19) after the calculation.

The operating system (OS) of the client device 30 used is “macOS Catalina” (trade name). The specifications of the processor (part of the control unit 31) were Quad Core Intel Core i7 (trade name) with a clock frequency of 3.5 GHZ, and the specifications of the memory (part of the storage unit 32) were 32 GB, 1600 MHZ, and DDR3. The specification of the graphics board was 4 GB of NVIDIA Geforce GTX 780 M (trade name), and the occupied data amount in the used Solid State Drive (SSD) was 190 GB (809.7 GB available/999.86 GB).

As described above, since the analysis result is displayed in a short time, the user can immediately know the analysis result. Even when the simulation is repeated a plurality of times while changing the parameter value, the total required time can be kept short. Therefore, it is easy for the user to change the parameter value and repeat the simulation a plurality of times. Then, the time required for designing and selecting the membrane reactor 1 can be shortened.

Furthermore, the simulation program is provided from the server device 20 of the service provider to the client device 30 of the user. Therefore, the user does not need to create a simulation program using the spreadsheet software by himself/herself, and can easily perform the simulation. The user can then concentrate on studying the membrane reactor 1 itself.

Further, the user can perform the simulation only by inputting the parameter values in the input cell 1 column 52 to the input cell 3 column 56 of the parameter input screen 50 (FIG. 8). In addition, in the input cell 1 column 52 to the input cell 3 column 56 of the parameter input screen 50 (FIG. 7), the variation factors for which the user should input the parameter values are partially limited. Further, in the input cell 1 column 52 to the input cell 3 column 56 of the parameter input screen 50 (FIG. 8), the input cell of the variation factor to which the user should input the parameter value is highlighted. In addition, the name of the variation factor to which the user should input the parameter value is displayed next to (on the left side of) the input cell. All of these matters are elements for enabling the user to easily perform the simulation without receiving special training for use of the simulation program.

Furthermore, according to the development support system 10 of the present exemplary embodiment, the simulation program is provided from the server device 20 to the client device 30 via the communication network CN (FIG. 2). Therefore, it is easy to develop the provision of the service related to the design and selection of the membrane reactor on the web. Then, the user can independently design and select the membrane reactor without inquiring the supplier of the product of the membrane reactor 1 about information such as a method of calculating a variation factor one by one. Furthermore, it is possible for the user to analyze the design and selection of the membrane reactor by himself/herself without the need of assistance of a supplier of the membrane reactor.

Such a point leads to reduction in personnel required for responding to an inquiry from a customer. For example, such an advantage is particularly effective for a supplier that conducts business with a relatively small number of personnel, such as a so-called startup company.

Furthermore, a large number of client devices 30 can individually perform simulation for one server device 20. Therefore, many simulations can be efficiently executed, and the product market of the membrane reactor 1 and the development support service market of the membrane reactor 1 can be activated. Then, productivity in the industrial column related to the membrane reactor 1 can be improved.

Here, the development support method according to the present embodiment is executed using the development support program executed by the server device 20 or the client device 30, and both the computer program executed by the server device 20 and the computer program executed by the client device 30 can be collectively regarded as the development support program. Furthermore, each of the computer program executed by the server device 20 and the computer program executed by the client device 30 can be regarded as a development support program.

### <Another Embodiment According to Calculation Step S3>

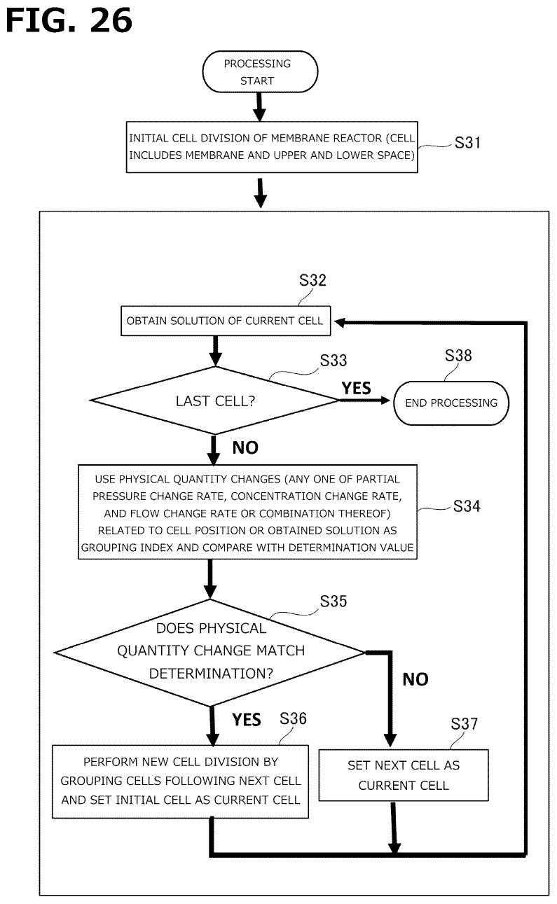

FIG. 26 schematically illustrates another embodiment according to the above-described calculation step (S21 to S25 in FIGS. 4 and 18). The method illustrated in FIG. 26 can shorten the processing time in the calculation step S3. The processing illustrated in FIG. 26 can be replaced with the processing of S21 to S25 in FIG. 18.

In FIG. 26, when the processing is started, the initial cell division related to the membrane reactor 1 is performed (S31). The initial cell division is, for example, processing of dividing the first unit cell (unit cell at the inlet 4) 2 in ten billion unit cells 2. That is, as illustrated in S21 of FIG. 18, not the entire cell division is performed at the beginning of the calculation step S3, but only a part of the unit cells (here, one unit cell) 2 is divided.

Here, an aspect of cell division is not limited to an aspect in which cells are divided by being arranged in a line as illustrated in FIG. 17, for example, and may be an aspect (not illustrated) in which cells are divided into a plurality of upper and lower columns in FIG. 17, for example. In addition, the mode of cell division is not limited to the mode of two-dimensional division, and may be a mode of three-dimensional division.

Subsequently, the flow formulas (various theoretical formulas using nonlinear multivariable simultaneous equations) are sequentially solved in each cell from the cell on the input side to the output side of the membrane reactor 1. Specifically, a solution is obtained for the current unit cell 2 (S32), and when the current unit cell 2 is the last unit cell 2 (S33: YES), the processing illustrated in FIG. 26 is ended after the predetermined post-processing is ended (S38).

In S33, when the current unit cell 2 is not the last unit cell 2 (S33: NO), the physical quantity change is compared with the determination value (S34). The physical quantity change relates to the cell position (the current position of the unit cell 2) or the obtained solution. Specifically, the physical quantity change is obtained from a solution of a flow formula (theoretical formula) based on [Mathematical Formula 1], [Mathematical Formula 2], or the like. Examples of the physical quantity change include the partial pressure change rate, the concentration change rate, the flow change rate, and the like. The physical quantity change is used as an index (grouping index) of the “grouping”. The determination value can be determined by knowledge (or by trial and error or the like) of the supplier that provides the application program. A plurality of determination values can be used to classify the determination values (for example, the determination value of a certain section division may be made different from the determination values of other section divisions.).

In the subsequent S35, it is determined whether or not the physical quantity change matches the determination, and when the physical quantity change matches the determination (when the change amount is smaller than the determination value) (S35: YES), the next new unit cell 2 is divided, and the divided new unit cell 2 is set as the current cell. Thereafter, the processing returns to S32.

In S35, when the physical quantity change does not match the determination (S35: NO), the next unit cell 2 is set as the current unit cell 2, and the processing returns to S32.

It can be said that the processing including S32 to S38 is processing of grouping the unit cells in accordance with the characteristics of the membrane reactor 1.

### <Specific Aspect of S32 (Calculation for Current Unit Cell 2)>

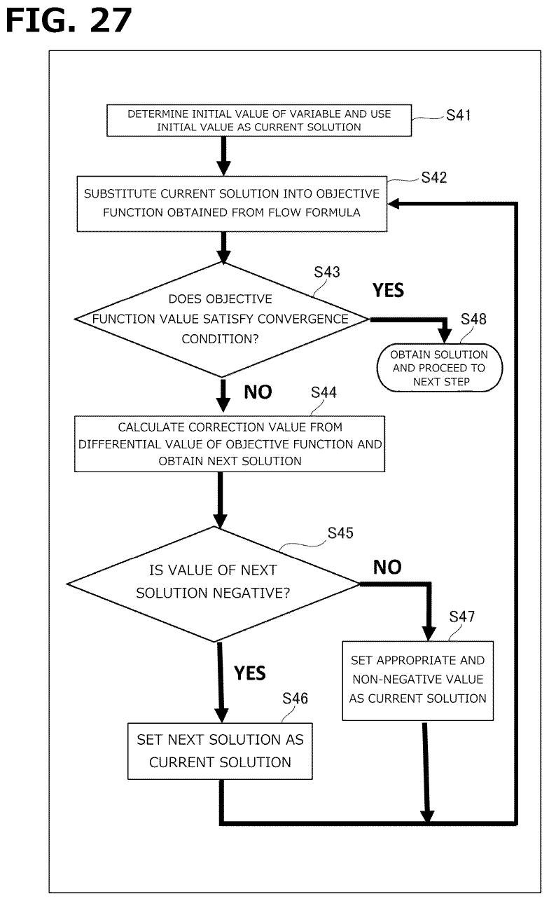

FIG. 27 illustrates an example of the processing (S32) of performing the calculation on the current unit cell 2 in FIG. 26. In FIG. 27, a case where the value of the solution is negative is handled. In FIG. 27, after S31 (initial cell division related to membrane reactor 1) in FIG. 26, the initial value of the variable is determined and used as the current (at that time) solution (S41).

Subsequently, in S42, the current solution (S41) is substituted into the objective function obtained from the flow formula. A determination is made as to whether the objective function satisfies the convergence condition (S43). When the objective function satisfies the convergence condition (S43: YES), a solution is obtained, and the processing proceeds to the next step (S26) in the calculation display processing (FIG. 18) (S48).

In S43, when the objective function does not satisfy the convergence condition (S43: NO), a differential value correction value of the objective function is calculated, and the next solution is obtained (S44). It is determined whether or not the value of the “next solution” obtained in S44 is not negative (S45), and in a case where the value is not negative (S45: YES), the “next solution” is set as the “current solution” (S46), and the processing returns to S42.

In S45, when the value of the “next solution” is negative (S45: NO), an appropriate value (non-negative value) is set as the current solution (S47), and the processing returns to S42.

### Others

Note that each of the above-described embodiments is merely an example of implementation in implementing the present invention, and the technical scope of the present invention should not be interpreted in a limited manner. That is, the present invention can be implemented in various forms without departing from the gist or main features thereof.

For example, the simulation target is not limited to development support of a membrane reactor for synthesizing methanol (MeOH) from carbon dioxide (CO₂) and hydrogen (H₂), and can also be applied to development support of a membrane reactor for other applications. The present invention can also be applied to support development of a membrane reactor in a reaction separation system that is strongly affected by chemical equilibrium, such as ammonia synthesis, dehydrogenation of methylcyclohexane, steam reforming of ethanol, steam reforming of methane, ester synthesis, and metanation.

### REFERENCE SIGNS LIST

1 Membrane reactor
2 Unit cell
3 Separation membrane
10 Development support system
20 Server device
30 Client device
50 Parameter input screen
52 Input cell 1 column
54 Input cell 2 column
56 Input cell 3 column
58 Result display cell 1 column
60 Result display cell 2 column
70 Membrane reaction module
72 Separation membrane element
74, 78 Container
80 Analysis result display screen
82 Goal seek screen
CN Communication network
P Reaction separation process step

1. A development support system comprising a service-provider-side terminal device and a user-side terminal device that are communicably connected,
wherein the service-provider-side terminal device determines whether or not a user of the user-side terminal device is a user satisfying a predetermined permission condition in a case where there is a request for use of a predetermined development support program from the user-side terminal device, and makes the development support program available for use to the user when it is determined that the user is the user satisfying the permission condition, and
at least a part of the development support program is described using an extended function language for giving an extended function to an application program with a calculation function installed in the user-side terminal device to cause the user-side terminal device to analyze a flow step of a membrane reactor that simultaneously performs a chemical reaction and membrane separation.

2. The development support system according to claim 1, wherein the user-side terminal device executes
a step of once equally dividing a series of reaction separation process steps into a first predetermined number of unit cells, and
a step of dividing a section from an inlet to an outlet of the series of reaction separation process steps into a plurality of sections that can be represented by the number of the unit cells and bundling the unit cells included in the divided sections into a second predetermined number smaller than the first predetermined number, and a step of calculating at least one of a mass balance and an energy balance of the unit cells by sequential calculation from the inlet to the outlet of the series of reaction separation process steps.

3. The development support system according to claim 2, wherein
in the sequential calculation, when the calculated value becomes a negative value although the calculated value needs to be physically a positive or zero numerical value, the user-side terminal device executes any one of convergence to the same value as the immediately preceding unit cell, convergence to zero, and convergence to a value that is infinitesimally small unless being zero.

4. The development support system according to claim 3, wherein
the user-side terminal device executes a step of displaying an analysis result, and
a time required to perform the sequential calculation and display the analysis result is 10 seconds or less.

5. The development support system according to claim 3, wherein
in the user-side terminal device, an input screen capable of individually changing a parameter value with respect to a predetermined membrane reactor characteristic and a variation factor of a predetermined reaction separation condition is displayed.

6. The development support system according to claim 5, wherein
in order to achieve a target analysis result, the user-side terminal device can execute a step of inversely calculating the value of the variation factor.

7. A development support device comprising a service-provider-side terminal device and a user-side terminal device that are communicably connected,
wherein the service-provider-side terminal device determines whether or not a user of the user-side terminal device is a user satisfying a predetermined permission condition in a case where there is a request for use of a predetermined development support program from the user-side terminal device, and
makes the development support program available for use to the user when it is determined that the user is the user satisfying the permission condition, and
at least a part of the development support program is described using an extended function language for giving an extended function to an application program with a calculation function installed in the user-side terminal device to cause the user-side terminal device to analyze a flow step of a membrane reactor that simultaneously performs a chemical reaction and membrane separation.

8. The development support device according to claim 7, wherein the user-side terminal device executes
a step of once equally dividing a series of reaction separation process steps into a first predetermined number of unit cells, and
a step of dividing a section from an inlet to an outlet of the series of reaction separation process steps into a plurality of sections that can be represented by the number of the unit cells and bundling the unit cells included in the divided sections into a second predetermined number smaller than the first predetermined number, and a step of calculating at least one of a mass balance and an energy balance of the unit cells by sequential calculation from the inlet to the outlet of the series of reaction separation process steps.

9. The development support device according to claim 8, wherein
in the sequential calculation, when the calculated value becomes a negative value although the calculated value needs to be physically a positive or zero numerical value, the user-side terminal device executes any one of convergence to the same value as the immediately preceding unit cell, convergence to zero, and convergence to a value that is infinitesimally small unless being zero.

10. The development support system according to claim 4, wherein
in the user-side terminal device, an input screen capable of individually changing a parameter value with respect to a predetermined membrane reactor characteristic and a variation factor of a predetermined reaction separation condition is displayed.

11. The development support system according to claim 10, wherein
in order to achieve a target analysis result, the user-side terminal device can execute a step of inversely calculating the value of the variation factor.
